(19) United States
(12) Patent Application Publication
(10) Pub. No.: US 2005/0114923 A1
(43) Pub. Date: May 26, 2005

(54) Plant biosensor systems

(75) Inventors: Michael Blaylock, Purcellville, VA (US); Bruce Ferguson, Great Falls, VA (US); Vicki L. Chandler, Tucson, AZ (US); Charissa Y. Poynton, London (GB)

Correspondence Address: CHOATE, HALL & STEWART LLP, EXCHANGE PLACE, 53 STATE STREET, BOSTON, MA, 02109, US

(73) Assignees: Edenspace Systems Corporation; University of Arizona

(21) Appl. No.: 10/882,632

(22) Filed: Jul. 1, 2004

Related U.S. Application Data
(60) Provisional application No. 60/483,891, filed on Jul. 1, 2003.

(51) Int. Cl. A01H 1/00; C12N 15/82; C12Q 1/26

(52) U.S. Cl. 800/282; 435/25; 435/468

(57) ABSTRACT

The invention provides a system for monitoring the bioavailability and/or toxicity of contaminants in the environment. The system includes plant biosensors that indicate the presence of contaminants, such as, for example, volatile organics or heavy metals, using an in situ monitoring approach. The plant biosensors are genetically engineered to exhibit a change in color phenotype under the control of promoter elements that are selectively responsive to the presence of a particular contaminant. The plant biosensing system provides a cost-effective approach to monitoring large and remote territories with high-spatial resolution. The transgenic plants can also be placed at strategic locations, before, during and after remedial activities at a site.

### RELATED APPLICATION

This application claims priority to Provisional Patent Application No. 60/438,891, filed on Jul. 1, 2003, which is incorporated herein by reference in its entirety.

### GOVERNMENT INTERESTS

Part of the work described herein was funded by the National Science Foundation (Award No. DMI-0320186). The United States Government may have certain rights in the invention.

### BACKGROUND OF THE INVENTION

Increasing concerns over the effects of chemicals in the environment and their toxicity and potential risks to human health have led to the necessity of monitoring pollutant levels at industrial, agricultural, residential, government and other sites.

Toxic metal ion pollution is perhaps one of the most difficult environmental problems, since unlike organic compounds, which can be degraded in the soil, metals are essentially non-mutable. Contamination with heavy metals has increased drastically due to industrialization, and heavy metals have reached toxic levels in the air, land, and water of many parts of the world (J. O. Nriagu, Environ. Pollut. 1988, 50: 139-161; J. O. Nriagu and J. M. Pacyna, Nature, 1988, 333: 134-139). Heavy metals such as cadmium, chromium, copper, lead, arsenic, mercury, nickel and zinc can be found contaminating urban sites, as well as agricultural areas. Heavy metals accumulate in soil and water from mining operations, industrial manufacturing facilities, recycling plants, and solid waste disposal locations. Furthermore, wind and/or rain can sometimes disperse metal contaminants at great distances from their points of use, production, or disposal, which significantly increases the possibility of endangering wildlife or ecosystems not initially affected. Detection and monitoring of heavy metals in the environment is not only important to remediate, control and minimize pollution and contamination associated with their accumulation, but can also help increase metal recovery/recycling efforts.

Traditionally, analysis of contaminants in the environment has been carried out in laboratories after sampling of the suspected polluted area. Samples of air, water or soil taken from the site under investigation are generally tested for the presence of pollutants using sophisticated techniques, such as, for example, atomic absorption spectrometry, ion chromatography, and inductively coupled plasma mass spectrometry (A. L. Burlingame et al., Anal. Chem. 1996, 68: 231R-256R; K. W. Jackson and G. Chem, Anal. Chem. 1996, 68: 231R). These techniques are often expensive, labor-intensive, time-consuming and not suitable for continuous field monitoring. They also have the disadvantage of only detecting the total amount of heavy metals and not the bioavailable concentrations accessible to living organisms (V. J. G. Houba et al., Sci. Total Environ. 1996, 178: 21-28; A. C. C. Plett et al., Environ. Toxicol. Chem. 1999, 18: 1882-1890; R. Badilla-Ohlbaum et al., Environ. Toxicol. Chem. 2001, 20: 2749-2757). Recent field-portable sensing devices such as x-ray fluorescence spectrometers provide faster readings, but also only measure total metal concentrations at a given point in time.

Alternative methods based on the use of field-portable biosensing systems have been developed that allow on-site detection of contamination and remote environmental monitoring. These systems exploit macromolecules, cells, tissues, or organisms in combination with electrochemical, optical, piezoelectric or thermometric transducers. Different types of such biosensors have, for example, been described for the detection of heavy metals. Whole cells (e.g., bacteria, fungi, lichens, mosses), enzymes (H. C. Budnikow et al., J. Electroanal. Chem. 1991, 310: 49-55; S. Fennouh et al., Biosens. Bioelectron. 1998, 13: 903-909; R. B. Thompson et al., Anal. Chem. 1998, 70: 4717-4723), apoenzymes (B. Mattiasson and H. Nilsson, J. Appl. Biochem. 1979, 1: 377-384; I. Satoh, Biosens. Bioelectron. 1991, 6: 375-379; I. Satoh, Ann. NY Acad. Sci. 1992, 672: 240-244), antibodies (M. Yhosraviane et al., Environ. Sci. Tech. 1998, 32: 137-142; D. A. Blake et al., Anal. Chim. Acta, 2001, 444: 3-11; I. A. Darwish and D. A. Blake, Anal. Chem. 2001, 73: 1889-1895), or proteins (I. Bontidean et al., Anal. Chem. 1998, 70: 4162-4169; I. Bontidean et al., J. Inorg. Biochem. 2000, 79: 225-229) have been used as recognition elements coupled to potentiometric, amperometric, optic, conductometric or spectrophotometric transducers. However, biosensing devices based on whole cells and enzymes have been shown to exhibit limited selectivity and low sensitivity, while systems based on metal binding proteins are highly sensitive but poorly selective as they were found to detect most heavy metal ions.

71 Transgenic organisms containing a reporter gene whose expression is controlled by a contaminant-specific responsive promoter have recently been introduced as a promising class of selective bio-indicators. Bacteria have been modified to make them synthesize an easily detectable product in the presence of a specific contaminant. Engineered bacteria exist for the detection of different organic chemicals such as benzene, polychlorinated biphenyls (PCBs), and naphthalene. Several microbial biosensors for the detection of various heavy metals have also been developed based on this approach (as reviewed in S. Ramanathan et al., Trends Biotechnol. 1997, 15: 500-506). Although these biosensing systems are fast, less expensive and less labor-intensive than other traditional methods, and provide physiologically relevant data in response to a specific contaminant, they exhibit several disadvantages including short lifetime, lack of genetic stability (which leads to variability in the response of different cells), and practical difficulties in field application such as masking by vegetation or soil.

Clearly, there is a need for improved methods to evaluate the level, bloavailability and/or toxicity of contaminants in environmental media, identify the trends of pollution, and control the efficiency of remediation activities. In particular, inexpensive and sensitive systems that can be widely and easily used, and that are capable of on-line operation, real-time detection and measurement of specific contaminants allowing direct assessment of their effects and associated risks to ecosystems and human health are highly desirable.

### SUMMARY OF THE INVENTION

The present invention provides an improved approach for environmental monitoring. In particular, the invention encompasses systems and strategies that allow rapid, cost-effective and accurate detection, identification and quantification of environmental contaminants (i.e., pollutants ranging from volatile organics to toxic heavy metals as well as useful micronutrients and valuable metals or metalloids). More specifically, transgenic plants are provided that indicate contamination of a site by a specific pollutant through a change in their color phenotype. In certain preferred embodiments, the inventive plant biosensors are genetically engineered to express high levels of anthocyanins under the control of promoter elements that are selectively and specifically responsive to the presence of a particular contaminant. The transgenic plants provided herein allow the bioavailable concentrations of contaminants to be determined in either the air, soil or water of natural, industrial, agricultural, residential, government or urban environments, and therefore can be used in a wide variety of environmental applications. For example, the inventive plant biosensors may be used in the monitoring of large and/or remote territories, in the pre-remediation assessment and post-remediation control of contaminated sites, in the detection of air pollutants, as biomarkers of toxic materials in drinking or irrigation waters, or in the detection of chemical and/or biological warfare materials.

In one aspect, the present invention provides nucleic acid constructs useful in the preparation of such transgenic plants. An inventive nucleic acid construct comprises a gene encoding a product involved in the biosynthesis of a plant pigment, said gene being operably linked to an inducible sensor system which is responsive to the presence of a contaminant. The sensor system preferably comprises a contaminant-responsive promoter element.

In certain embodiments, the gene encodes a product involved in the biosynthesis of anthocyanins. For example, the gene encodes an anthocyanin regulatory gene B, an anthocyanin regulatory gene R, or translational equivalents thereof.

In certain embodiments, the contaminant comprises an inorganic compound or element. For example, the contaminant may comprise a metal or a metalloid, such as a heavy metal selected from the group consisting of antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium and zinc. The contaminant may comprise an inorganic anion such as chloride, perchlorate, nitrate, phosphate or sulfate, or a gaseous inorganic compound such as ozone, a sulfur oxide or a nitrogen oxide.

In other embodiments, the contaminant comprises an organic compound. For example, the contaminant may comprise a volatile organic compound (VOC), such as benzene or formaldehyde, or a halogenated organic compound such as a polychlorinated biphenyl (PCB), a polychlorinated hydrocarbon (PCH), a chlorofluorocarbon (Freon), or a chlorofluoro-hydrocarbon, or may be selected from the group consisting of a dioxin, a furan, a polycyclic aromatic hydrocarbon and an ether.

In still other embodiments, the contaminant may comprise a pesticide.

In yet other embodiments, the contaminant may comprise an agent of chemical or biological warfare, such as sarin, ricin, mustard gas, phosgene, anthrax bacilli, anthrax spores, or smallpox virus.

In certain embodiments, induction of the sensor system causes expression of the gene, for example, increased expression of the gene, in response to the presence of the contaminant. In those embodiments where induction of the sensor system causes increased expression of the gene, the increase expression is preferably proportional to the concentration of contaminant present, more preferably proportional to the concentration of bioavailable contaminant present.

In other embodiments, induction of the sensor system causes increased expression of the gene in response to the presence of the contaminant. Preferably, the increase in expression is proportional to the concentration of contaminant present.

In still other embodiments, the nucleic acid construct further comprises a sequence selected from the group consisting of a leader sequence, an intron sequence, a transcription terminator, a polyadenylation site, and any combinations thereof. In yet other embodiments, the nucleic acid construct further comprises a sequence selected from the group consisting of a marker gene sequence, a selectable gene sequence, a T-DNA sequence, and any combinations thereof.

In another aspect, the present invention provides transgenic plant cells stably transformed with one or more inventive nucleic acid constructs. A transgenic plant cell of the invention preferably comprises a transgene comprising a gene encoding a product involved in the biosynthesis of a plant pigment, said gene being operably linked to an inducible sensor system, wherein the sensor system is responsive to the presence of a contaminant.

In certain embodiments, the transgenic plant cell is from a monocotyledonous plant species. In other embodiments, the transgenic plant cell is from a dicotyledonous plant species. The cell may be from a plant selected from the group consisting of turfgrasses, members of the Brassicaceae family, ferns of the Pteris genus of the Pteridaceae family, members of the Nicotiana genus of the Solanacea family, and members of the Poaceae family. In particular, the cell may be from Maize or an Arabidopsis. Alternatively or additionally, the cell may be from a crop plant, a landscape plant or a house plant.

In another aspect, the present invention provides a transgenic plant exhibiting a change in color phenotype when exposed to a contaminant. Preferred transgenic plants are plants transformed with an inventive nucleic acid construct described herein. In certain embodiments, the transgenic plant comprises a gene encoding a product involved in the biosynthesis of a plant pigment, said gene being operably linked to an inducible sensor system which is responsive to the presence of a contaminant.

Exposure of the transgenic plant to the contaminant causes induction of the sensor system, which results in expression of the gene, for example increased expression of the gene, and a change in color phenotype of the plant. Preferably, the increase in expression of the gene is proportional to the concentration of contaminant present, more preferably, to the concentration of bioavailable contaminant present.

Also provided by the present invention are a seed of any generation of the inventive transgenic plant; progeny of the inventive transgenic plant; and plant material from the inventive transgenic plant, wherein the seed, plant progeny and plant material each comprise the transgene.

The present invention also provides a transgenic plant that carries multiple transgenes for simultaneous sensing of multiple contaminants, the transgenic plant exhibiting different changes in color phenotype when exposed to different contaminants. In certain embodiments, the transgenic plant comprises more than one transgenes, each transgene comprising a gene encoding a product involved in the biosynthesis of a plant pigment, said gene being operably linked to an inducible sensor system which is responsive to the presence of a particular contaminant and whose induction by that particular contaminant is associated with a specific change in color phenotype. 1025] In another aspect, the present invention provides a method for preparing a transgenic plant exhibiting a change in color phenotype when exposed to a contaminant using an inventive nucleic acid construct described herein. In certain embodiments, the method comprises: introducing a nucleic acid construct into a plant cell or protoplast to obtain a stably transformed plant cell or protoplast; regenerating a whole plant from the stably transformed plant cell or protoplast to obtain a transgenic plant; and exposing the transgenic plant to the contaminant, wherein the contaminant is present in the air, in water or in a soil.

Stably transformed plant cells, stably transformed protoplasts and transgenic plants obtained using the inventive methods are provided by the present invention. Also provided are seeds, progeny and plant material from transgenic plants obtained by the inventive method of transformation.

In yet another aspect, the present invention provides methods for detecting the presence and/or level of one or more than one contaminant in an area.

A preferred method for detecting the presence of a contaminant in an area comprises: introducing, in the area to be tested, seeds from a transgenic plant of the invention; growing transgenic plants from the seeds; monitoring the color phenotype from the resulting plants; and determining that the contaminant is present in the area tested if a change in color phenotype was observed in the resulting plants.

In certain embodiments, introducing seeds comprises planting the seeds in the soil of the area to be tested. In other embodiments, introducing seeds comprises planting the seeds in potting soil or growth media to obtain seedlings, and introducing the seedlings to the area to be tested. In still other embodiments, the transgenic plants are grown in water.

In those embodiments where the increase in expression of the gene is proportional to the concentration of contaminant or bioavailable contaminant, the method further comprises assessing the intensity of change in color phenotype of the resulting plants; and, based on the intensity, determining the concentration of contaminant or bioavailable contaminant present in the area.

A preferred method for detecting the presence and/or level of more than one contaminant in an area comprises: introducing, in the area to be tested, seeds from a transgenic plant comprising more than one transgene, each transgene comprising: a gene encoding a product involved in the biosynthesis of a plant pigment, said gene being operably linked to an inducible contaminant and whose induction by that particular contaminant is associated with a specific change in color phenotype; growing transgenic plants from the seeds; monitoring the color phenotype of the resulting plants; and determining that a particular contaminant is present in the area tested if the associated change in color phenotype was observed in the resulting plants.

Other aspects, features and advantages of the present invention will become apparent from the following detailed description. It should be understood, however, that the detailed description and specific examples, while indicating preferred embodiments of the invention, are given by way of illustration only.

### BRIEF DESCRIPTION OF THE DRAWING

FIG. 1 shows a map of the plasmid, pMCG7216, used for the transformations described in Examples 3 and 4.

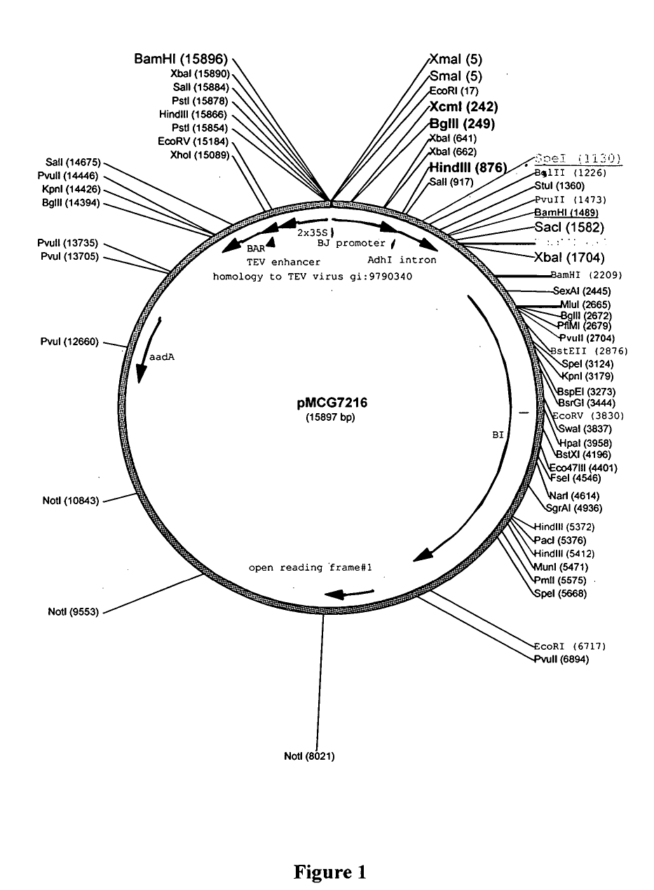

### DEFINITIONS

Throughout the specification, several terms are employed that are defined in the following paragraphs.

As used herein, the term “nucleic acid construct” refers to a polynucleotide or oligonucleotide comprising nucleic acid sequences not normally associated in nature. A nucleic acid construct of the present invention is prepared, isolated, or manipulated by the hand of man. The terms “nucleic acid”, “polynucleotide” and “oligonucleotide” are used herein interchangeably and refer to a deoxyribonucleotide (DNA) or ribonucleotide (RNA) polymer either in single- or double-stranded form. For the purposes of the present invention, these terms are not to be construed as limited with respect to the length of the polymer and should also be understood to encompass analogs of DNA or RNA polymers made from analogs of natural nucleotides and/or from nucleotides that are modified in the base, sugar and/or phosphate moieties.

As used herein, the term “gene” refers to a discrete nucleic acid sequence responsible for a discrete cellular product and/or performing one or more intracellular or extracellular functions. More specifically, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. The gene and regulatory sequences may be derived from the same natural source, or may be heterologous to one another. The definition can also include nucleic acids that do not encode proteins but rather provide templates for transcription of functional RNA molecules such as tRNAs, rRNAs, etc. Alternatively, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA) or a protein produced by translation of an mRNA. Gene products also include RNAs that are modified by processes such as capping, polyadenylation, methylation, and editing, proteins post-translationally modified, and proteins modified by, for example, methylation, acetylation, phosphorylation, ubiquitination, ADP-ribosylation, myristilation, and glycosylation.

In the context of the present invention, the term “product involved in the biosynthesis of a plant pigment” refers to a gene product (e.g., primary transcript, mRNA, pre-protein, protein, or higher order complex) that participates in the intracellular biochemical reactions that ultimately result in the production of a plant pigment.

As used herein, the term “operably linked” refers to a relationship between two nucleic acid sequences wherein the expression of one of the nucleic acid sequences is controlled by, regulated by or modulated by the other nucleic acid sequence. Preferably, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species.

In the context of the present invention, the terms “inducible sensor system” and “sensor system” are used interchangeably. They refer to a polynucleotide which is, in some manner, dependent upon an external stimulus (such as the presence of a contaminant) in order to actively transcribe and/or translate a gene to which it is operably linked. A sensor system is “responsive to the presence of a contaminant” or “induced by a contaminant” when the contaminant acts as an external stimulus for the sensor system. A sensor system may comprise one or more of a number of sensory and regulatory entities, such as, for example, promoters, regulatory elements, enhancers, regulatory proteins, antisense RNA, transport and receptor proteins and other parts of a signal transduction machinery. Preferred sensor systems of the invention comprise contaminant-responsive promoter elements.

In the context of the present invention, the term “contaminant” refers to any compound, molecule, agent or entity whose presence and/or level in an area or environment is to be detected. The term encompasses compounds, molecules, agents or entities which may be considered as harmful as well as compounds, molecules, agents or entities which may be considered as useful or valuable. A contaminant may be present in the air, in water or in a soil. In certain embodiments of the invention, the contaminant is a compound or elemental form of a metal or metalloid, preferably a heavy metal, such as antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium or zinc. In other embodiments, the contaminant is a volatile organic compound (or VOC), such a benzene or formaldehyde. In still other embodiments, the contaminant is an organic compound such as a polychlorinated biphenyl (PCB), polychlorinated hydrocarbon (PCH), methyl tertiary butyl ether (MTBE) or various pesticides. In yet other embodiments of the invention, the contaminant is a compound or elemental form of an inorganic, non-metallic substance, such as chloride, perchlorate, nitrate, phosphate, sulfate, or ozone. In other embodiments of the invention, the contaminant is an agent of chemical or biological warfare, such as sarin, ricin, mustard gas, anthrax bacilli or spores, or smallpox virus.

As used herein, the terms “promoter” and “promoter element” refer to a polynucleotide that regulates expression of a selected polynucleotide sequence operably linked to the promoter, and which effects expression of the selected polynucleotide sequence in cells. A “contaminant-responsive promoter element” is a promoter whose activity modulates in response to the presence and/or level of a contaminant. The term “plant promoter”, as used herein, refers to a promoter that functions in a plant. Constitutive promoters as well as tissue-specific promoters which selectively function in a part of a plant body, including a flower, are preferable. Examples of plant promoters include, but are not limited to, Cauliflower mosaic virus (CaMV) 35S promoter and a promoter of nopaline synthase.

In the context of the present invention, the term “increased expression of a gene” refers to a gene activation that results in an increase in production of a gene product. Gene activation includes those processes that increase transcription of a gene and/or translation of an mRNA. Gene activation can constitute, for example, inhibition or repression as well as stimulation of expression above an existing level. In embodiments of the present invention that relate to transgenic plants, an increased expression of a gene results in a change in the color phenotype of the plants. Preferably, the increase in expression of the gene is proportional to the concentration of contaminant present. More preferably, the increase in expression of the gene additionally correlates with the intensity of change in color phenotype of the transgenic plants. When the change in color phenotype relates to a change in color intensity of the plant, the increase in expression of a gene corresponds to an increase in production of a gene product by about 2-fold, preferably from about 2- to about 5-fold, or from about 5- to about 10-fold, more preferably from about 10- to about 20-fold, or from about 20- to about 50-fold, still more preferably from about 50- to about 100-fold, even more preferably 100-fold or more.

The term “transgene”, as used herein, refers to an exogenous gene which, when introduced into a host cell through the hand of man, for example, using a process such as transformation, electroporation, particle bombardment, and the like, is expressed by the host cell and integrated into the cell's DNA such that the trait or traits produced by the expression of the transgene is inherited by the progeny of the transformed cell. A transgene may be partly or entirely heterologous (i.e., foreign to the cell into which it is introduced). Alternatively, a transgene may be homologous to an endogenous gene of the cell into which it is introduced, but is designed to be inserted (or is inserted) into the cell's genome in such a way as to alter the genome of the cell (e.g., it is inserted at a location which differs from that of the natural gene or its insertion results in a knockout). A transgene can also be present in a cell in the form of an episome. A transgene can include one or more transcriptional regulatory sequences and other nucleic acids, such as introns. Alternatively or additionally, a transgene is one that is not naturally associated with the vector sequences with which it is associated according to the present invention.

As will be clear from the context, the term “plant”, as used herein, can refer to a whole plant, plant parts (e.g., cuttings, tubers, pollen), plant organs (e.g., leaves, stems, flowers, roots, fruits, branches, etc.), individual plant cells, groups of plant cells (e.g., cultured plant cells), protoplasts, plant extracts, seeds, and progeny thereof. The class of plants which can be used in the methods of the present invention is as broad as the class of higher plants amenable to transformation techniques, including both monocotyledonous and dicotyledonous plants, as well as certain lower plants such as algae. The term includes plants of a variety of a ploidy levels, including polyploid, diploid and haploid. In certain embodiments of the invention, the plant is selected from the group consisting of turfgrasses, members of the Brassicaceae family, ferns of the Pteris genus of the Pteridaceae family, members of the Nicotiana genus of the Solanacea family, and members of the Poaceae family. In many embodiments of the invention, the plant is Maize or an Arabidopsis. In other embodiments of the invention, the plant is selected from the group consisting of crop plants, landscaping plants and house plants.

As used herein, the term “protoplast” refers to an isolated plant cell without cell walls which has the potency for regeneration into cell culture or a whole plant.

As used herein, the term “transformation” refers to a process by which an exogenous nucleic acid molecule (e.g., a vector or recombinant DNA molecule) is introduced into a recipient cell, callus or protoplast. The exogenous nucleic acid molecule may or may not be integrated into (i.e., covalently linked to) chromosomal DNA making up the genome of the host cell, callus or protoplast. For example, the exogenous polynucleotide may be maintained on an episomal element, such as a plasmid. Alternatively, the exogenous polynucleotide may become integrated into a chromosome so that it is inherited by daughter cells through chromosome replication. Methods for transformation include, but are not limited to, electroporation, magnetoporation, Ca²⁺ treatment, injection, particle bombardment, retroviral infection, and lipofection.

As used herein, the term “stably transformed”, when applied to a plant cell, callus or protoplast refers to a cell, callus or protoplast in which an inserted exogenous nucleic acid molecule is capable of replication either as an autonomously replicating plasmid or as part of the host chromosome. The stability is demonstrated by the ability of the transformed cells to establish cell lines or clones comprised of a population of daughter cells containing the exogenous nucleic acid molecule.

The terms “genetically modified” and “transgenic” are used herein interchangeably. A transgenic or genetically modified organism is one that has a genetic background which is at least partially due to manipulation by the hand of man through the use of genetic engineering. For example, the term “transgenic cell”, as used herein, refers to a cell whose DNA contains an exogenous nucleic acid not originally present in the non-transgenic cell. A transgenic cell may be derived or regenerated from a transformed cell or derived from a transgenic cell. Exemplary transgenic cells in the context of the present invention include plant calli derived from a stably transformed plant cell and particular cells (such as leaf, root, stem, or reproductive cells) obtained from a transgenic plant. A “transgenic plant” is any plant in which one or more of the cells of the plant contain heterologous nucleic acid sequences introduced by way of human intervention. Transgenic plants typically express DNA sequences, which confer the plants with characters different from that of native, non-transgenic plants of the same strain. The progeny from such a plant or from crosses involving such a plant in the form of plants, seeds, tissue cultures and isolated tissue and cells, which carry at least part of the modification originally introduced by genetic engineering, are comprised by the definition.

As used herein, the term “color phenotype” or “color phenotypic trait” refers to a visually observable property or set of properties resulting from the expression or suppression of a gene or genes involved in the biosynthesis of a plant pigment. A change in color phenotype, when applied to a transgenic plant, refers to a change in color, color intensity and/or color pattern compared with the corresponding color, color intensity and/or color pattern of the native, non-transgenic plants of the same strain. A change in color may, additionally or alternatively, be a change in intensity, duration or spatial pattern of electromagnetic radiation emitted by the plant, or reflected by the plant, under natural (such as sunlight) or artificial (such as an ultraviolet lamp) conditions, at any wavelength or set of wavelengths.

As used herein, the term “regeneration” refers to the process of growing a plant from a plant cell (e.g., plant protoplast, plant callus or plant explant).

Additional definitions are provided throughout the Detailed Description.

### DETAILED DESCRIPTION OF CERTAIN PREFERRED EMBODIMENTS

The present invention relates to the detection and monitoring of contaminants in the environment. In particular, the invention relates to an improved approach that allows rapid, cost-effective and real-time detection and measurement of the levels of certain organic or inorganic contaminants in the air, water or soil of natural, industrial, agricultural, residential, government, urban or other environments. More specifically, the present invention is directed to systems including transgenic plants that indicate the presence of contaminants by a change in their color phenotype. In certain embodiments, the inventive plant biosensors are genetically engineered to express high levels of a plant pigment under the control of contaminant-responsive promoter elements. Preferably, the promoter element is selectively and specifically responsive to the presence of a particular contaminant.

Accordingly, in one aspect, the present invention provides nucleic acid constructs that are useful to obtain such transgenic plants.

I. Nucleic Acid Constructs

Nucleic acid constructs of the invention preferably comprise a gene encoding a product involved in the biosynthesis of a plant pigment, said gene being operably linked to an inducible sensor system, which is selectively responsive to the presence of a contaminant. In preferred embodiments, the gene encodes a product involved in the biosynthesis of anthocyanins.

Anthocyanin Regulatory and Biosynthetic Genes

The anthocyanins form a class of pigments that are responsible for the greater part of the red, purple, and blue colors in flowers and fruits of the higher plants. These pigments function to attract pollinating insects to plants and shield plant DNA from UV damage. From a chemical viewpoint, anthocyanin pigments belong to the class of flavanoids. In particular, the anthocyanins are glycosides the hydrolysis of which leads to the formation of sugars and colored aglycons (e.g., cyanidin, malvidin, peonidin, pelargonidin, etc). Examples of anthocyanins include, but are not limited to, pelargonidin glycosides occurring in geraniums; cyanidin-3-rutinoside occurring in, for example, snap-dragons; cyanidin-3-galactoside occurring in, for example, apples; malvidin glycosides, peonidin glycosides, delphinidin glycosides occurring in, for example, hydrangeas; and coumaryl petudinin glycosides occurring in, for example, grapes and petunias.

There are multiple anthocyanin genes that participate in the production of pigments that cause plants to display different colors. In corn or maize, anthocyanin biosynthesis requires expression of twenty or more genes. Some of these genes are anthocyanin biosynthetic genes; others are anthocyanin regulatory genes. An anthocyanin biosynthetic gene is a gene whose product is involved in the biosynthesis (i.e., production) of the anthocyanins. An anthocyanin regulatory gene is a gene whose products are regulatory proteins which activate the transcription of one or more anthocyanin biosynthetic genes. Examples of anthocyanin biosynthetic genes or loci in Maize include c2, chi, f3h, a1, a2, bronze1 (bz1) and bz2 (E. Coe et al., in “Corn and Corn Improvement”, G. Sprague and J. Dudley (Eds.), 1998, American Society of Agronomy: Madison, Wis.). Examples of anthocyanin regulatory genes or loci in Maize include R, B and Lc.

The expression of anthocyanin regulatory genes is complex (see Examples section). For example, more than fifty naturally occurring alleles of R that condition unique patterns of pigmentation have been described. It has been determined that all R genes encode functionally equivalent proteins and that developmental specificity of pigmentation is determined by differences in the R promoter region. It is now well known in the art that transcriptional activators of anthocyanin biosynthesis, operably linked to a suitable promoter in a construct, have widespread utility as non-phytotoxic markers for plant cell transformation.

Accordingly, in certain embodiments, the nucleic acid construct provided by the present invention comprises an anthocyanin regulatory gene or fragment thereof, which preferably comprises an R gene or B gene, or translational equivalents thereof. The nucleotide sequences of various R genes and B genes are available in Genbank. In one embodiment, the anthocyanin regulatory genes are derived from maize. Due to the inherent degeneracy of the genetic code, other polynucleotide sequences which encode substantially the same or a functionally equivalent product involved in the biosynthesis of anthocyanins also may be used in the inventive nucleic acid construct.

Inducible Sensor Systems

An inducible sensor system of the present invention is any polynucleotide, which is, in some manner, dependent upon an external stimulus, such as the presence of a contaminant, in order to actively transcribe and/or translate a gene to which it is operably linked.

In certain embodiments of the invention, the inducible sensor system comprises a contaminant-responsive promoter element. A contaminant-responsive promoter element for use in the practice of the present invention may be any polynucleotide that is selectively induced by a contaminant, causing expression of the gene to which the promoter is operably linked. Preferably, the promoter element is selectively and specifically responsive to a contaminant (i.e., it is induced by a single member or by a few members of a given class of contaminants and not by other members of the same class of contaminants or by members of a different class of contaminants).

In certain embodiments, the promoter element is responsive to organic contaminants. Organic contaminants include, but are not limited to, volatile organic compounds (VOCs), such as, for example, benzene, formaldehyde, toluene, xylenes and styrene; organo-halogenated compounds such as polychlorinated biphenyls (PCBs); ozone-depleting solvents, such as, for example, chlorofluorocarbons (e.g., Freon), methyl chloroform, carbon tetrachloride, and hydrochlorofluorocarbons; other toxic chemicals such as dioxins, furans, and polycyclic aromatic hydrocarbons; explosive or weapons components such as TNT; chemical or biological warfare agents such as sarin, ricin, phosgene, mustard gas, anthrax bacilli or spores, or smallpox virus.

In other embodiments, the promoter element is responsive to inorganic contaminants, such as compounds or elemental forms of metals or metalloids, preferably heavy metals. Heavy metals include antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium or zinc. Other inorganic contaminants include compounds or elemental forms of non-metallic substances such as chloride, perchlorate, nitrate, phosphate, sulfate, or ozone. In still other embodiments, the promoter element is responsive to radioactive isotopes.

Chemical-responsive promoters for use in plants have been reported (see, for example, C. Gatz, Ann. Rev. Plant Physiol. Plant Mol. Biol. 1997, 48: 89-108; C. Gatz, Nature Biotechnol. 1998, 16: 140), including steroid-inducible promoters (A. M. Lloyd et al., Science, 1994, 266: 436-439), estradiol-inducible promoters (W. Bruce et al., Plant Cell, 2000, 12: 65-79), and ethanol-inducible promoters (M. X. Caddick et al., Nat. Biotechnol. 1998, 16: 177-180).

Selective contaminant-responsive promoters may readily be identified by those of ordinary skill in the art. For instance, Examples 1 and 2 describe procedures through which selective contaminant-responsive promoter elements are identified by rapidly screening thousands of plant genes of commercially available DNA microarrays. Examples of such DNA microarrays include, but are not limited to, the Arabidopsis GeneChip, which contains approximately 25,000 genes of the well-characterized laboratory plant, Arabidopsis thalia tia, and the Barley Genome GeneChip™, both from Affymetrix (Santa Barbara, Calif.). Screening may also be conducted using customized arrays.

Using commercially available or customized DNA (micro)arrays, comparative genomic hybridization analyses can performed using RNA extracted from leaves of plants exposed to a specific contaminant and from plants grown under identical conditions except for the presence of contaminant. Genes that are up-regulated in the presence of the contaminant can be identified as potentially useful as bio-indicators. Additional studies carried out by exposing plants to different contaminants may help pinpoint those genes that are selectively responsive to a contaminant of interest. Following the selection of promising candidate contaminant-responsive genes, the 5′ (promoter) of each gene may be identified and used as sensor system in the inventive nucleic acid construct.

As is well-known in the art, a nucleic acid construct may preferably contain other appropriate regulatory signals. Accordingly, in certain embodiments of the invention, in addition to the sensor system and anthocyanin regulatory or biosynthetic gene, the nucleic acid construct also comprises other polynucleotide sequences, such as leader sequences, intron sequences, transcription terminators, polyadenylation sites, and any combinations thereof.

Other Polynucleotide Sequences

An inventive nucleic acid construct may contain a plant promoter, for example, a constitutive plant promoter. Examples of plant promoters include, but are not limited to, the 35S cauliflower mosaic virus (CaMV) promoter, a promoter of nopaline synthase and a promoter of octopine synthase. Examples of other constitutive promoters used in plants are the 19 S promoter, and promoters from genes encoding actin or ubiquitin. The promoters may be obtained from genomic DNA by using polymerase chain reaction (PCR), and then cloned into the construct.

Other sequences that can be present in a nucleic acid construct of the instant invention are sequences that enhance gene expression, such as intron sequences and leader sequences. Examples of introns that have been reported to enhance expression include, but are not limited to, the introns of the Maize Adh1 gene and introns of the Maize bronze1 gene (J. Callis et. al., Genes Develop. 1987, 1: 1183-1200). Examples of non-translated leader sequences that are known to enhance expression include, but are not limited to, leader sequences from Tobacco Mosaic Virus (TMV, the “omegasequence”), Maize Chlorotic Mottle Virus (MCMV), and Alfalfa Mosaic Virus (AIMV) (see, for example, D. R. Gallie et al., Nucl. Acids Res. 1987, 15: 8693-8711; J. M. Skuzeski et. al., Plant Mol. Biol. 1990, 15: 65-79).

Optional components of the nucleic acid construct include a marker gene. Marker genes are genes that impart a distinct phenotype of cells expressing the marker gene and thus allow transformed cells to be distinguished from cells that do not have the marker. Such genes may encode either a selectable or screenable marker. Many examples of suitable marker genes are known in the art and can be used in the practice of the invention including a dominant herbicide resistance gene or the bar gene which codes for the enzyme phosphinothricin acetyl transferase.

The nucleic acid construct of the invention may be cloned into a vector, such as, for example, a plasmid. Vectors suitable for transforming plant cells include, but are not limited to, Ti plasmids from Agrobacterium tumefaciens (J. Darnell, H. F. Lodish and D. Baltimore, “Molecular Cell Biology”, 2^{nd} Ed., 1990, Scientific American Books: New York.), a plasmid containing a β-glucuronidase gene and a cauliflower mosaic virus (CaMV) promoter plus a leader sequence from alfalfa mosaic virus (J. C. Sanford et al., Plant Mol. Biol. 1993, 22: 751-765) or a plasmid containing a bar gene cloned downstream from a CaMV 35S promoter and a tobacco mosaic virus (TMV) leader. Other plasmids may additionally contain introns, such as that derived from alcohol dehydrogenase (Adh1), or other DNA sequences. The size of the vector is not a limiting factor.

For constructs intended to be used in Agrobacterium-mediated transformation, the plasmid may contain an origin of replication that allows it to replicate in Agrobacterium and a high copy number origin of replication functional in E. coli. This permits facile production and testing of transgenes in E. coli prior to transfer to Agrobacterium for subsequent introduction in plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

Methods of preparation of nucleic acid constructs and expression vectors are well known in the art and can be found described in several textbooks such as, for example, J. Sambrook, E. F. Fritsch and T. Maniatis, “Molecular Cloning: A Laboratory Manuar”, 1989, Cold Spring Harbor Laboratory: Cold Spring Harbor, and T. J. Silhavy, M. L. Berman, and L. W. Enquist, “Experiments with Gene Fusions”, 1984, Cold Spring Harbor Laboratory: Cold Spring Harbor; F. M. Ausubel et al., “Current Protocols in Molecular Biology”, 1989, John Wiley & Sons: New York.

II. Method of Preparation of Transgenic Plant Biosensor Systems

The nucleic acid constructs described above can be used to transform any plant. Accordingly, the present invention provides methods of preparation of transgenic plants that exhibit a change in color phenotype when exposed to a particular contaminant. Using the inventive methods of transformation, genetically modified plants, plant cells, plant tissue, seeds, and the like can be obtained.

A preferred method comprises steps of introducing a nucleic acid construct provided herein into a plant cell or protoplast to obtain a stably transformed plant cell or protoplast; and regenerating a whole plant from the stably transformed plant cell or protoplast.

Cell Transformation

Delivery or introduction of a nucleic acid construct into eukaryotic cells may be accomplished using any of a variety of methods. The method of transformation is not critical to the instant invention. Suitable techniques include, but are not limited to, non-biological methods, such as microinjection, microprojectile bombardment, electroporation, induced uptake, and aerosol beam injection, as well as biological methods such as direct DNA uptake, liposomes and Agrobacterium-mediated transformation. Any combinations of the above methods that provide for efficient transformation of plant cells or protoplasts may also be used in the practice of the invention.

Methods of introduction of nucleic acid constructs into plant cells or protoplasts have been described. See, for example, “Methods for Plant Molecular Biology”, Weissbach and Weissbach (Eds.), 1989, Academic Press, Inc; “Plant Cell, Tissue and Orgall Culture: Fundamental Methods”, 1995, Springer-Verlag: Berlin, Germany; and U.S. Pat. Nos. 4,945,050; 5,036,006; 5,100,792; 5,240,855; 5,302,523; 5,322,783; 5,324,646; 5,384,253; 5,464,765; 5,538,877; 5,538,880; 5,550,318; 5,563,055; and 5,591,616).

In particular, electroporation has frequently been used to transform plant cells (see, for example, U.S. Pat. No. 5,384,253). This method is generally performed using friable tissues (such as a suspension culture of cells or embryogenic callus) or target recipient cells from immature embryos or other organized tissue that have been rendered more susceptible to transformation by electroporation by exposing them to pectin-degrading enzymes or by mechanically wounding them in a controlled manner. Intact cells of Maize (see, for example, K. D'Halluin et al., Plant cell, 1992, 4: 1495-1505; C. A. Rhodes et al., Methods Mol. Biol. 1995, 55: 121-131; and U.S. Pat. No. 5,384,253), wheat, tomato, soybean, and tobacco have been transformed by electroporation. As reviewed, for example, by G.W. Bates (Methods Mol. Biol. 1999, 111: 359-366), electroporation can also be used to transform protoplasts.

Another method of transformation is microprojectile bombardment (see, for example, U.S. Pat. Nos. 5,538,880; 5,550,318; and 5,610,042; and WO 94/09699). In this method, nucleic acids are delivered to living cells by coating or precipitating the nucleic acids onto a particle or microprojectile (for example tungsten, platinum or gold), and propelling the coated microprojectile into the living cell. Microprojectile bombardment techniques are widely applicable, and may be used to transform virtually any monocotyledonous or dicotyledonous plant species (see, for example, U.S. Pat. Nos. 5,036,006; 5,302,523; 5,322,783 and 5,563,055; WO 95/06128; A. Ritala et al., Plant Mol. Biol. 1994, 24: 317-325; L. A. Hengens et al., Plant Mol. Biol. 1993, 23: 643-669; L. A. Hengens et al., Plant Mol. Biol. 1993, 22: 1101-1127; C. M. Buising and R. M. Benbow, Mol. Gen. Genet. 1994, 243: 71-81; C. Singsit et al., Transgenic Res. 1997, 6: 169-176).

The use of Agrobacterium-mediated transformation of plant cells is well known in the art (see, for example, U.S. Pat. No. 5,563,055). This method has long been used in the transformation of dicotyledonous plants, including Arabidopsis and tobacco, and has recently also become applicable to monocotyledonous plants, such as rice, wheat, barley and maize (see, for example, U.S. Pat. No. 5,591,616). In plant strains where Agrobacterium-mediated transformation is efficient, it is often the method of choice because of the facile and defined nature of the gene transfer. Agrobacterium-mediated transformation of plant cells is carried out in two phases. First, the steps of cloning and DNA modifications are performed in E. coli, and then the plasmid containing the gene construct of interest is transferred by conjugation or electroporation into Agrobacterium, and the resulting Agrobacterium strain is used to transform plant cells.

Transformation of plant protoplasts can be achieved using methods based on calcium phosphate precipitation, polyethylene glycol treatment, electroporation, and combinations of these treatments (see, e.g., I. Potrykus et al., Mol. Gen. Genet. 1985, 199: 169-177; M. E. Fromm et al., Nature, 1986, 31: 791-793; J. Callis et al., Genes Dev. 1987, 1: 1183-1200; S. Omirulleh et al., Plant Mol. Biol. 1993, 21: 415-428).

Alternative methods of plant cell transformation, which have been reviewed, for example, by M. Rakoczy-Trojanowska (Cell Mol. Biol. Lett. 2002, 7: 849-858), can also be used in the practice of the present invention.

The successful delivery of the nucleic acid construct into the host plant cell or protoplast may be preliminarily evaluated visually. Selection of stably transformed plant cells can be performed, for example by introducing into the cell, a nucleic acid construct comprising a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Examples of herbicides which may be used include phosphinothricin and glyphosate. Potentially transformed cells then are exposed to the selective agent. Cells where the resistance-conferring gene has been integrated and expressed at sufficient levels to permit cell survival will generally be present in the population of surviving cells.

Alternatively, host cells comprising a nucleic acid sequence of the invention and which express its gene product may be identified and selected by a variety of procedures, including, DNA-DNA or DNA-RNA hybridizations and protein bioassay or immunoassay techniques such as membrane, solution or chip-based technologies for the detection and/or quantification of nucleic acid or protein.

Plant cells are available from a wide range of sources including the American Type Culture Collection (Rockland, Md.), or from any of a number of seed companies including, for example, A. Atlee Burpee Seed Co. (Warminster, Pa.), Park Seed Co. (Greenwood, S.C.), Johnny Seed Co. (Albion, Me.), or Northrup King Seeds (Hartsville, S.C.). Descriptions and sources of useful host cells are also found in I. K. Vasil, “Cell Culture and Somatic Cell Genetics of Plants”, Vol. I, II and II; 1984, Laboratory Procedures and Their Applications Academic Press: New York; R. A. Dixon et al., “Plant Cell Culture—A Practical Approach”, 1985, IRL Press: Oxford University; and Green et al., “Plant Tissue and Cell Culture”, 1987, Academic Press: New York.

Plant cells or protoplasts stably transformed with an inventive nucleic acid construct are provided by the present invention. Stably transformed plant cells or protoplasts comprise a transgene comprising a gene encoding a product involved in the biosynthesis of a plant pigment, preferably anthocyanins, operably linked to a sensor system which is responsive to the presence of a contaminant.

Plant Regeneration

In plants, every cell is capable of regenerating into a mature plant, and in addition contributing to the germ line such that subsequent generations of the plant will contain the transgene of interest. Stably transformed cells may be grown into plants according to conventional ways (see, for example, McCormick et al., Plant Cell Reports, 1986, 5: 81-84). Plant regeneration from cultured protoplasts has been described, for example by Evans et al., “Handbook of Plant Cell Cultures”, Vol. 1, 1983, MacMilan Publishing Co: New York; and I. R. Vasil (Ed.), “Cell Culture and Somatic Cell Genetics of Plants”, Vol. I (1984) and Vol. II (1986), Acad. Press: Orlando.

Means for regeneration vary from species to species of plants, but generally a suspension of transformed protoplasts or a Petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. Glutamic acid and proline may also be added to the medium. Efficient regeneration generally depends on the medium, on the genotype, and on the history of the culture.

Regeneration from transformed individual cells to obtain transgenic whole plants has been shown to be possible for a large number of plants. For example, regeneration has been demonstrated for dicots (such as apple; Malus pumila; blackberry, Rubus; Blackberry/raspberry hybrid, Rubus; red raspberry, Rubus; carrot; Daucus carota; cauliflower; Brassica oleracea; celery; Apium graveolens; cucumber; Cucumis sativus; eggplant; solanum melongena; lettuce; Lactuca sativa; potato; Solanum tuberosum; rape; Brassica napus; soybean (wild); Glycine Canescens; strawberry; Fragaria x ananassa; tomato; Lycopersicon esculentum; walnut; Juglans regia; melon; Cucumis melo; grape; Vitis vinifera; mango; and Mangifera indica) as well as for monocots (such as rice; Oryza sativa; rye, Secale cereale; and Maize).

Primary transgenic plants may then be grown using conventional methods. Various techniques for plant cultivation are well known in the art. Plants can be grown in soil, or alternatively can be grown hydroponically (see, for example, U.S. Pat. Nos. 5,364,451; 5,393,426; and 5,785,735). Primary transgenic plants may be either pollinated with the same transformed strain or with a different strain and the resulting hybrid having the desired phenotypic characteristics identified and selected. Two or more generations may be grown to ensure that the subject phenotypic characteristics is stably maintained and inherited and then seeds are harvested to ensure that the desired phenotype or other property has been achieved.

As is well known in the art, plants may be grown in different media such as soil, growth solution or water.

Selection of plants that have been transformed with the construct may be performed by any suitable method, for example, with Northern blot, Southern blot, herbicide resistance screening, antibiotic resistance screening or any combinations of these or other methods. The Southern blot and Northern blot techniques, which test for the presence (in a plant tissue) of a nucleic acid sequence of interest and of its corresponding RNA, respectively, are standard methods (see, for example, Sambrook & Russell, “Molecular Cloning”, 2001, Cold Spring Harbor Laboratory Press: Cold Spring Harbor).

III. Transgenic Plants

In another aspect, the present invention provides transgenic plants that indicate the presence of a contaminant through a change in their color phenotype. The change in color phenotype may be a change in color, in color intensity, and/or in color pattern.

More specifically, the present invention provides a transgenic plant that comprises a transgene comprising a gene encoding a product involved in the biosynthesis of a plant pigment, said gene being operably linked to an inducible sensor system which is responsive to the presence of a contaminant. Preferably, the gene encodes a product involved in the biosynthesis of anthocyanins.

The present invention also provides a transgenic plant that comprises multiple transgenes for simultaneous sensing of multiple contaminants, wherein each transgene comprises a sensor system which is responsive to the presence of a different contaminant, and wherein the induction of each sensor system is associated with a different change in color phenotype. The changes in color phenotype may be changes in color, changes in color intensity, and/or changes in color patterns.

In these embodiments, the term “change in color” refers to a change in intensity, duration or spatial pattern of electromagnetic radiation emitted by the plant, or reflected by the plant, under natural (such as sunlight) or artificial (such as an ultraviolet lamp) conditions, at any wavelength or set of wavelengths.

In certain embodiments, the plant is a monocotyledonous plant. In other embodiments, the plant is a dicotyledonous plant. Preferably, the plant is selected from the group consisting of turfgrasses, members of the Brassicaceae family, ferns of the Pteris genus of the Pteridaceae family, members of the Nicotiana genus of the Solanacea family, and members of the Poaceae family. In particular, the plant may be Maize or an Arabidopsis. The plant may a crop plant, a landscaping plant or a house plant. The choice of the plant will depend on the intended purpose of the transgenic plant.

Additional desirable properties of the transgenic plants may include, but are not limited to, fast growth (growth cycle of 6 to 8 weeks); ability to adapt for growth in various climates and soil conditions; well studied genetic model system; incorporation of bioconfinement features such as male sterile flowers; incorporation of phytoremediation features such as contaminant hyperaccumulation, greater biomass, or promotion of contaminant-degrading mycorrhizae.

IV. Uses of Transgenic Plants

In another aspect, the present invention provides methods of use of the transgenic plants as biosensing systems. In particular, methods are provided that allow detection, identification and determination of the levels of contaminants in natural, industrial, agricultural, government or urban environments. Detection and quantification of specific contaminants according to the methods of the instant invention may be carried out whether the contaminant is present in the air, soil or water of the environment under investigation.

00] A preferred method for detecting the presence of a contaminant in an area comprises steps of introducing, in the area to be tested, seeds from a transgenic plant of the invention; monitoring the color phenotype of the resulting plants; and determining that a particular contaminant is present in the area if a change in color phenotype is observed in the resulting plants. In certain embodiments, the method further comprises steps of assessing the intensity of change in color phenotype of the resulting plants; and, based on the intensity measured, determining the concentration of contaminant in the area tested.

A preferred method for detecting the presence and/or level of more than one contaminant in an area comprises: introducing, in the area to be tested, seeds from a transgenic plant comprising more than one transgene, each transgene comprising: a gene encoding a product involved in the biosynthesis of a plant pigment, said gene being operably linked to an inducible contaminant and whose induction by that particular contaminant is associated with a specific change in color phenotype; growing transgenic plants from the seeds; monitoring the color phenotype of the resulting plants; and determining that a particular contaminant is present in the area tested if the associated change in color phenotype was observed in the resulting plants.

The methods of the invention allow the bioavailable concentrations of contaminants to be determined in either the air, soil or water of natural, industrial, agricultural, residential, government or urban environments, and therefore can be used in a wide variety of environmental applications. For example, the inventive plant biosensors may be used in the monitoring of large and/or remote territories, in the pre-remediation assessment and post-remediation control of contaminated sites, in the detection of air pollutants, as biomarkers of toxic materials in drinking or irrigation waters, or in the detection of chemical and/or biological warfare materials. A few examples illustrating their possible uses are detailed below.

Detection of Arsenic, Cadmium and Lead in Drinking Water

Contamination of drinking water by arsenic, cadmium, and lead is widespread and poses significant health risks to millions of people worldwide. All three elements are introduced into water supplies not only as a result of industrial, agricultural and manufacturing processes, but also through erosion of natural depots. Consequently, their presence in the environment is all but inevitable, making speedy, accurate detection of their presence in drinking water an important part of reducing health risks.

Arsenic is a known carcinogen and mutagen, and is detrimental to the immune system. Ingestion of inorganic arsenic, the primary form in drinking water, can result in cancer (e.g., skin, bladder, and prostate) and other health problems (e.g., thickening of the skin, effects on the nervous system, hearing impairment, cardiovascular effects, mutations and birth defects, gastrointestinal system and liver effects, development of diabetes). Arsenic is introduced in water supplies through wood preservatives, agricultural chemicals such as herbicides and pesticides, semiconductor manufacturing, and other industrial uses.

Short-term exposure to cadmium can cause nausea, muscle cramps, sensory disturbances, liver injury, shock and renal failure. Longer-term exposure can result in damage to kidneys, liver, bone and blood. Cadmium co-occurs naturally with zinc, lead, copper and other ores, which can serve as sources to ground and surface waters, particularly when in contact in soft acidic waters. In the U.S., cadmium released in the environment was primarily from metal smelting and refining. Cadmium can be introduced into drinking water through corrosion of some galvanized plumbing and water main pipe materials.

In 1991, the Secretary of Heath and Human Services called lead “the number one environmental threat to the health of children in the United States”. While people of all ages are at risk from lead poisoning, children and pregnant women are especially vulnerable. Lead exposure can impair a child's mental and physical development, reduce a baby's birth weight, and cause premature birth. Prolonged exposure can cause aggressive behavior, hyperactivity and learning problems. In adults, increased lead levels have been linked to kidney problems, high blood pressure and damaged hearing. Serious lead poisoning can lead to blindness, brain damage and mental retardation. The source of most lead in drinking water provided by public water systems is pipe or solder used in home plumbing. Water supplied not treated in public water systems may contain lead from soil contamination caused by agricultural use of chemicals containing lead, leaded gasoline, lead paint, industry or mining, or erosion of natural deposits.

The primary method for measuring concentrations of arsenic, cadmium, and lead in drinking water is by laboratory analysis of water samples. In the case of arsenic, for example, tests often use volatilization of arsine as in the Marsch test or Gutzeit modification, or a colorimetric method base on the reaction of arsine with diethyldithiocarbamate. Samples are usually collected either by professional inspectors or by consumers of the water. Costs range from as low as $15 to $30 for a single test for lead (excluding sampling costs), to approximately $150 for a single test for a suite of contaminants. While analysis by certified laboratories provides an accurate measure of contaminant concentrations in the samples, these methods measure point-in time contaminant levels rather than cumulative exposures.

08 J The use of transgenic plants of the invention as biomarkers for arsenic, cadmium and lead can provide low-cost cumulative monitoring of toxic heavy metals in drinking water. Plants use water in evapotranspiration. As a result, plant biosensing system should demonstrate low sampling variability because of continuous collection of data over long periods of time. Transgenic plants are also expected to offer greater convenience and lower costs than current sampling and analysis protocol.

Detection of Volatile Organic Compounds

Volatile organic compounds (VOCs) include hydrocarbons such as ethers, esters, ketones, aldehydes, and alcohols. Industrial plants, which release these types of organic compounds or solvents into the atmosphere, include, for example, semi-conductors, paint manufacturing, fiberglass molding operations, dry cleaning, auto and appliance paint baking and textiles.

Certain VOCs such as, for example, benzene and formaldehyde, are classified as air toxics, known or suspected to cause cancer. In September 2000, the U.S. Environmental Protection Agency (USEPA) issued a Report to Congress (“National Air Toxics Program: The Integrated Urban Strategy”), which summarized risk assessments and research needs associated with 33 urban air toxics, i.e., air pollutants known, or suspected, to cause cancer and other serious health effects, particularly in urban areas. These toxic gases are a subset of the 188 toxic air pollutants identified under the Clean Air Act. In 1989, the USEPA reported to Congress that indoor air pollution poses serious acute and chronic health risks to a major portion of the public (Volume I: Executive summary and recommendations, EPA/400/1-89/001a; Volume II: Assessment and control of indoor air pollution, EPA/400/1-89/001c) and has been identified by scientists at the Mayo Clinic as the likely cause for a tripling of asthma cases in children aged 1 to 14 from Rochester, Minn. Indoor air quality problems are often caused by typical building equipment and furnishings, such as photocopiers, paints, ceiling tiles, and floor covering, which emit organic volatile compounds such as benzene and formadehyde, particularly when these items are new.

Exposure to air toxics can pose significant health risks, particularly for children. Airborne benzene, for example, can cause leukemia with an estimated risk of four cases per million among people having a lifetime exposure to benzene concentrations greater than 1 μg/m³ in air (R. Rinsky et al., Am. J. Ind. Med. 1981, 2: 217-245; G. G. Bond et al., Br. J. Ind. Med. 1986, 43: 685-691; World Health Organization, Updating and Revision of the Air Quality Guidelines for Europe, 1996, Report No. EUR/ICP/EHAZ9405/MT 121996) and “urban benzene” has recently been identified as the cause for deteriorating indoor air quality in several large cities in Europe, with urban and house levels of about 20 μg/m³ and 10 μg/m³, respectively in Athens, Greece (V. Cocheo et al., Nature, 2000, 44: 142-144). The U.S. National Institute for Occupational Safety and Health has set the Immediately Dangerous to Life and Health (IDLH) concentration of benzene at 1.6 μg/m³ (NIOSH, “Pocket Guide to Chemical Hazards”, US Govt Print Office, 1997). The major sources of environmental benzene are tobacco smoke, stored fuels and paint supplies, and automobile emissions.

Similarly, formaldehyde at concentrations as low as 0.1 ppm contributes to sick building syndrome and is a suspected carcinogen. The U.S. National Institute for Occupational Safety and Health has set the IDLH concentration for formaldehyde at 24.6 mg/m³ (NIOSH, 1997). Major sources of environmental formaldehyde include pressed wood products (e.g., plywood paneling, particleboard, fiberboard), furniture made with these pressed wood products , glues, and tobacco smoke.

13] The two most common commercial methods of detecting airborne VOCs are with photo-ionization detectors (PIDs) and with high-temperature metal oxide semi-conductor (HMOS) sensors. PIDs are more expensive, generally costing more than $1,000 for a portable unit, but can be calibrated to detect more than 100 VOCs in a range of 0-10,00 ppm with a resolution of about 1% and response time of less than 5 seconds. The more common technology, HMOS, makes use of the changes in electrical resistance in a metal oxide in the presence of different gases to detect VOCs. The least expensive portable HMOS detectors can cost less then $200, but their range (>5 ppm) and resolution (>10%) are significantly worse than for PIDS.

Other technologies include the use vapor-sensitive dyes known as metalloporphyrins to detect VOCs. By imaging the dyes with a scanner or camera before and after exposure to a VOC, color changes in the dyes can be detected. Although this technology should be adaptable to the detection of hazardous VOCs, it is not yet commercially available in sensor products. Therefore, current available techniques are highly sensitive but cost-prohibitive (PIDs) or too poorly sensitive to be useful in the detection of VOCs in urban and residential areas.

Transgenic plants of the present invention, in particular those engineered to contain a transgene comprising a gene encoding a product involved in the biosynthesis of anthocyanin, operably linked to a VOC-responsive promoter element, may be used as low-cost environmental sensors to detect specific VOCs at threshold levels that would be harmful to small children. The main advantage of the inventive method is the possibility it offers to detect toxic airborne chemicals using common houseplants. The plant biomarker method is expected to provide greater convenience and lower costs that PID and HMOS sensors, particularly in schools and homes. As a “threshold” detection method, it will lack the resolution of current technologies, instead providing an indication of contamination above a certain level so that action may be taken, ranging from standard air sampling and analysis, to remediation or intervention. The change in color phenotype of the transgenic plants used as VOC detectors may also provide strong psychological incentives to reduce VOC levels. Another significant advantage of plant biosensors is the ability to scale up the observation area at low cost. Public parks, playgrounds, and residential yards, for example, could be planted with the biosensors, providing three-dimensional maps of air quality—and human exposure to toxic chemicals—for little more than landscaping costs.

Detection of Heavy Metals

Heavy metals include antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium or zinc.

Small amounts of these elements, or some forms of them, are commonly found in foodstuffs, fruits, vegetables, and commercially available multivitamin products and are actually nutritionally essential for a healthy life. Some of these elements, such as iron, copper, manganese, and zinc, are referred to as the trace elements. Heavy metals become toxic when they are not metabolized by the body and accumulate in the soft tissues. Heavy metal toxicity can result in damaged or reduced mental and central nervous function, lower energy levels, and damage to blood composition, lungs, kidneys, liver and other vital organs. Long-term exposure may result in slowly progressing physical, muscular, and neurological degenerative processes that mimic Alzheimer's disease, Parkinson's disease, muscular dystrophy, and multiple sclerosis. Allergies are not uncommon and repeated long-term contact with some metals or their compounds may even cause cancer. Heavy metals may enter the human body through food, water, air or absorption through the skin when they come in contact with humans in agriculture and in manufacturing, pharmaceutical, industrial or residential settings. Industrial exposure accounts for a common route of exposure of adults. Ingestion is the most common route of exposure in children. As a rule, acute poisoning is more likely to result from inhalation or skin contact of dust, fumes or vapors, or materials in the workplace. However, lesser levels of contamination may occur in residential settings, particularly in older homes with lead paint or old plumbing.

Heavy metals have found applications in various fields. For example, diagnostic medical applications include direct injection of gallium during radiological procedures, dosing with chromium in parenteral nutrition mixtures, and the use of lead as a radiation shield around x-ray equipment. Heavy metals are also common in industrial applications such as in the manufacture of fertilizers, pesticides, batteries, alloys, electroplated metal parts, dyes, steel, etc.

Monitoring heavy metals in the environment, particularly in large or remote areas, is often cost-prohibitive due to the expense of the extensive sampling required to adequately assess heterogeneous distribution of the contaminants.

Plant biosensors of the invention that indicate the presence of specific heavy metals through a change in color phenotype can offer high spatial resolution, stand-off reporting, ready scaling to large areas, and continuous operation. Improving the ability to accurately monitor and assess heavy metal contaminations will improve awareness of contaminated areas and provide a low cost assessment of private sites by homeowners, farmers, and industry. Of particular usefulness will be the ability of farmers to detect the potential bioavailability of heavy metals to food crops as a result of heavy metals in soil applied biosolids and fertilizer materials. Transgenic plants of the invention can also assist in detecting and monitoring heavy metals in landfill leachate, runoff and migration of surface soil contamination, providing indications of metal availability useful for risk assessments. Plant biosensors can also be placed at strategic locations, before, during and after remedial activities at a contaminated site allowing real-time evaluation of the performance of remediation systems.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

The examples described below take advantage of the anthocyanin biosynthetic pathway, which has been used as a visual marker by plant geneticists for 80 years. Extensive studies in four plant species (maize, Petunia, Arabidopsis, and Antirrhinum) have unraveled the regulation of the anthocyanin pathway (for a review, see, for example, J. Mol et al., Trends in Plant Sciences, 1998, 3: 212-217). In specific genetic background, introduction of a single transcription factor can result in activation of the pathway, and the amount of pigment produced is a sensitive indicator of the amount of the transcription factor present. Where and when the transcription factor is activated can be under the control of a heterologous promoter that responds to signals of interest. Importantly, the transcription factors are usually rate limiting; thus, the amount of pigment observed is proportional to the transcriptional activation of the gene encoding the transcription factor. The anthocyanin biosynthetic pathway has therefore excellent potential to provide a sensitive, readily visible biosensor in plants.

Control of Anthocyanin Biosynthesis

The control of anthocyanin biosynthesis has been studied in both the Maize aleurone layer of the kernel and in seedlings, juvenile and mature plant tissues. All tissues require the biosynthetic genes c2, chi, f3h, a1, a2, bronze1 (bz1) and bz2 (E. Coe et al., in “Corn and Corn Improvement”, G. Sprague and J. Dudley (Eds.), 1998, American Society of Agronomy: Madison, Wis.). Where and when these biosynthetic genes are activated depends on the developmental and tissue-specific regulation of several transcription factors.

Activation of the transcription factors is mediated at the transcriptional level by distinct promoter elements unique for each allele of the regulatory genes (C. Tonelli et al., Mol. Gen. Genet. 1991, 225: 401-410; J. P. Radicella et al., Genes & Dev. 1992, 7: 2152-3264; E. L. Walker et al., EMBO J. 1995, 14: 2350-2363; G. I. Patterson et al., Genetics, 1995, 140: 1389-1406; D. A. Selinger et al., Genetics, 1998, 149: 1125-1138; B. May and S. L. Dellaporta, Plant J. 1998, 13: 241-247; and K. Petroni et al., Genetics, 2002, 155: 323-336). Molecular studies have shown that the b and r genes produce homologous basic-helix-loop-helix (bHLH) transcription factors (S. R. Ludwig et al., Proc. Natl. Acad. Sci. USA, 1989, 86: 7092-7096; V. L. Chandler et al., Plant Cell, 1989, 1: 1175-1183), which are functionally interchangeable (S.R. Ludwig et al., Science, 1990, 247: 449-450; S. A. Goff et al., EMBO J. 1990, 9: 2517-2522). The b and r gene products directly interact with the c1 and pl1 gene products, which are functionally interchangeable myb transcription factors (J. Paz-Ares et al., EMBO J. 1986, 5: 829-833; K. C. Cone et al., Plant Cell, 1993, 5: 1795-1805), to induce the expression of the biosynthetic genes (S. A. Goff et al., Genes Dev. 1992, 6: 864-875). Expression of c1 is limited to the seed, while the functionally equivalent protein encoded by pl1 is primarily expressed in seedlings and plant tissues (K. C. Cone et al., Plant Cell, 1993, 5: 1795-1805). The b1 and r1 genes have multiple alleles, each conferring a specific pattern of tissue-specific anthocyanin production (E. Styles et al., Can. J. Genet. Cytol. 1973, 15: 59-72). Characterization of several B and Pl1 alleles, which are transcribed at different levels, has demonstrated that the amount of anthocyanin produced is proportional to the transcriptional level of the regulatory gene (G. I. Patterson et al., Genetics, 1993, 135: 881-894; J. B. Hollick et al., Genetics, 2000, 154: 1827-1838), suggesting that the expression of these factors is under the control of a responsive promoter.

The anthocyanin biosynthetic genes have multiple elements close to the transcription start site required for co-activation by the bHLH factor encoded by b or r together with the myb-like factor encoded by c1 or pl1 (B. A. Roth et al., Plant Cell, 1991, 3: 317-325; J .A. Tuerck and M. E. Fromm, Plant Cell, 1994, 6: 1655-1663; J. P. Bodeau and V. Walbot, Plant Mol. Biol. 1996, 32: 599-609; M. B. Sainz et al., Plant Cell, 1997, 9: 611-625; M. L. Lesnick and V. L. Chandler, Plant Physiol. 1998, 117: 437-445). C1 binds to some of these elements (M. B. Sainz et al., Plant Cell, 1997, 9: 611-625), but these promoters share a sequence required for activation that does not bind to C1 (M. L. Lesnick and V. L. Chandler, Plant Physiol. 1998, 117: 437-445).

A number of investigators have successfully activated anthocyanin synthesis in other plant species by fusing the regulatory genes to heterologous promoters and introducing them into the species of interest (see, for example, A. M. Lloyd et al., Science, 1992, 258: 1773-1775; F. Quattrocchio et al., Plant Cell, 1993, 5: 1497-1512; Z. Z. Gong et al., Plant Mol. Biol. 1999, 41: 33-44). Whether one or both transcription factors are required for activation depends on the genotype of the recipient species. For example, in Arabidopsis and tobacco, introduction of the bHLH factor is sufficient to activate anthocyanin accumulation (A. M. Lloyd et al., Science, 1992, 258: 1773-1775). Transgenic Maize plants have been generated that contain either a BcDNA clone or the B genomic region spanning the transcribed region fused to the 35SCaMV promoter (Chandler laboratory—unpublished data). The resulting transgenic plants are purple in young seedlings and throughout their vegetative and floral tissues, with the exception of a few tissues, such as anthers, where it is known that the 35SCaMV promoter is not expressed.

In addition to Maize, extensive genetic and molecular dissection have been carried out on the anthocyanin pathway in several other plant species. Studies from Petunia and Arabidopsis revealed similarities and differences with Maize. In Petunia, bHLH proteins encoded by an1 and JAF13 (C. Spelt et al., Plant Cell, 2000, 12: 1619-1632; F. Quattrocchio et al., Plant J. 1998, 13: 475-488), myb proteins encoded by an2 (F. Quattrocchio et al., Plant Cell, 1999, 11: 1433-1444), and a WD40 protein encoded by anl1 (N. de Vetten et al., Genes Dev. 1997, 11: 1422-1434) are implicated in anthocyanin gene regulation. In Arabidopsis, the bHLH protein appears to be encoded by TT8 (N. Nesi et al., Plant Cell, 2000, 12: 1863-1878), the myb protein by TT2 (N. Nesi et al., Plant Cell, 2001, 13: 2099-2114), and the WD40 protein by TTG1 (A. R. Walker et al., Plant Cell, 1999, 11: 1337-1350). Another gene, anl2 (anthocyaninless2; encoding a Glabra2-related homeodomain protein) shows anthocyanin defects in all leaf and root subepidermal tissues, but has no effect on seed pigmentation (H. Kubo et al., Plant Cell, 1999, 11: 1217-1226). Anl2 could be acting to specify subepidermal cell identity.

While there are similar themes in the regulatory circuits in all species, there are three interesting differences between regulation of the pathway in Maize as compared to these dicot species (J. Mol et al., Trends in Plant Sciences, 1998, 3: 212-217). First, in Maize the regulatory proteins control steps throughout the biochemical pathway from the first step (encoding by c2, chalcone synthase) through the last two steps, encoded by bz1 and bz2. In contrast, in the dicot species the orthologous regulatory proteins do not activate the first three steps of the pathway (chalcone synthase, chi and f3h); the first gene activated encodes DFR, which is encoded by a1 in Maize. A second interesting difference is that mutations in these three classes of regulatory proteins in Maize, the bHLH, myb or WD40 (D. A. Selinger and V. L. Chandler, Plant Cell, 1999, 11: 5-14), have no pleiotropic developmental or growth phenotypes. The only detectable phenotype is lack of anthocyanin pigments. In contrast, mutations in these genes in Arabidopsis result in alterations in epidermal cell patterning in the roots and leaves, mucilage and anthocyanins in the seed coat and in the formation of root hairs and trichomes (see, for example, D. B. Szymanski et al., Trends Plant Sci. 2000, 5: 214-219; T. L. Western et al., Plant Physiol. 2000, 122: 345-356; Y. Lin and J. Schiefelbein, Development, 2001, 128: 3697-3705). Several aspects of this have been investigated and there is good evidence that TTG1 activates several regulatory genes involved in trichome patterning (A. Schnittger et al., Plant Cell, 1999, 11: 1105-1116; Y. Lin and J. Schiefelbein, Development, 2001, 128: 3697-3705). Some of these developmental phenotypes are mediated by interactions between anthocyanin regulatory proteins with other regulatory proteins specifying the developmental pathways (C. T. Payne et al., Genetics, 2000, 156: 1349-1362). A third difference is the expression analyses of the regulatory proteins. In Maize, the regulatory proteins do not regulate the expression of themselves or each other (S. A. Goff et al., EMBO J. 1990, 9: 2517-2522; D. A. Selinger and V. L. Chandler, Plant Cell, 1999, 11: 5-14). In contrast, in Petunia, ectopic expression of the AN2 encoded myb protein results in increased expression of an1 that is also dependent on the other myb protein, an4, in anthers (C. Spelt et al., Plant Cell, 2000, 12: 1619-1632).

### Example 1

### Transgenic Plants For the Detection of Volatile Organic Compounds

The experiments described herein are designed to determine the feasibility of using plant biomarkers to detect volatile organic compounds (VOCs) such as benzene and formaldehyde. First, all of the approximately 25,000 genes of a well-characterized laboratory plant, Arabidopsis thaliana, are screened to identify those genes that are up-regulated by benzene and formaldehyde.

Identification of Arabidopsis Genes up-regulated by Benzel ne and Forinaldehyde

Arabidopsis thaliana plants are exposed for either one hour or twenty-four hours to benzene and formaldehyde at a high dose (equilibrium concentration at saturation, i.e., at its vapor pressure) in an isolated chamber where the concentration of VOC can be measured. Leaf tissues are then harvested after exposure to VOC. Total RNA from these tissues is isolated and used to analyze the level of mRNA in each treated sample. An Affymetric whole genome Arabidopsis micro-array chip, which contains about 25,000 Arabidopsis genes, is used to monitor the expression level of each gene. Hybridization is carried out using a dual color labeling protocol in which one color represents the untreated sample and the other color represents the treated sample. A comparison of the intensities of the two colors allows identification of genes that are up-regulated by the VOC at a specific dose and a specific time point. Bioinfor mnatics tools such as GeneSpring software are used to conduct quality control and to analyze the expression profile data. It is anticipated that hundreds of genes will be identified as up-regulated by each VOC. A major milestone will be the identification of a small number (about 5 to 10) of genes that are expressed only in the presence of a VOC at the tested concentration.

Arabidopsis plants for VOC exposure: Arabidopsis plants are grown in 16 hours of white light and 8 hours of dark regime at a constant temperature of 23° C., which are the standard Arabidopsis laboratory growth conditions. Liquid fertilizer is supplied to the plants to maintain optimum growth. Arabidopsis seeds are germinated by placing the seeds in opaque plastic trays with water and aerated solutions.

Exposure ofArabidopsis Plants to Selected VOCs: Four-week old Arabidopsis plants are transferred to a transparent, sealed chamber. The chamber (1 m³) has a removable top and is fitted with a gasket, bolts and wing nuts to provide an airtight seal. The top of each chamber contains two small removable ports (0.25 inch in diameter), through which contaminants can be introduced and air samples can be collected. The Plexiglas chamber is located in a controlled environmental growth room equipped with central ventilation with an activated carbon filter system installed in the ventilation outlet to trap any leakage of benzene and formaldehyde from the test chamber. The growth room is maintained at a 16 hours light/8 hours dark, 23° C. regime with light intensity at plant height of 300 μmol m⁻² s⁻¹.

Benzene or formaldehyde is injected into the chamber to reach the desirable concentration level. Before introducing VOCs into the Plexiglas chamber, the airtight seal of the chamber is checked by injecting ammonia hydroxide into the chamber. Only if the chamber has a completely airtight seal is it used for the experiments. To introduce formaldehyde into the Plexiglas chamber with or without plants, formaldehyde solution (37% of formaldehyde) is pumped into the chamber by a gas scrubbing apparatus filled with an appropriate amount of formaldehyde solution. The gas scrubbing apparatus is attached to both an air pump and to the chamber-sampling inlet using Tygon tubing. Air is pumped into the formaldehyde solution and introduced into the Plexiglas chamber as gas. A small electric fan is installed inside the chamber to mix the air. For benzene contaminated air, an appropriate amount of benzene is injected into a small metal tray attached to the Plexiglas chamber wall just below the introduction port and is allowed to evaporate with the helps of the fan into the chamber. A period of 30 minutes is allowed for complete evaporation and mixing of the VOCs inside the chamber before collecting initial air samples. For each sample, 500 mL of air are collected from the chambers into stainless steel tubes (18 cm long by 0.6 cm diameter). The air samples are analyzed using a DuPont GC Mass Spectrometer.

Seven to nine plants are used for treatment and seven to nine plants are used for controls. To ensure the quality and reliability of the experiment, this process is repeated. The plants are then removed, and leaf tissues are placed in liquid nitrogen in preparation for shipment before being analyzed.

Isolation and Analysis of RNA: The leaves from VOC treated plants are used for RNA isolation using the hot phenol/lithium chloride method (see, for example, “Current Protocols in Molecular Biology”) and analyzed by gel electrophoresis for quality. Total RNA is isolated following the protocol described by C. Puissant and L. M. Houbebine (Biotech. 1990, 8: 148-149) Approximately 5 to 10 g of Arabidopsis plants are ground to fine powder in liquid nitrogen in a chilled mortar and pestle. The ground tissue is placed in 4 to 6 Falcon 2059 centrifuge tubes (Becton Dickinson, Lincoln Park, N.J.) each containing 5 mL of GuISCN extraction buffer (4 M guanidinium isothiocyanate, 25 mM sodium citrate (pH 7.0), 05% (w/v) N-lauroyl sarkosine and 0.1 M β-mercaptoethanol), and the tubes are mixed by inversion.

Samples are mixed with 0.1 volume of 2 M sodium acetate (pH 4.0) and 5 mL of phenol:chloroform (5:1) are added. Samples are then mixed and centrifuged at 5000 g for 15 minutes at 4° C. The aqueous phase (about 7 mL) is removed and placed in a fresh 15 mL Falcon 2059 tube and the RNA is precipitated by addition of an equal volume of isopropanol at 4° C. The RNA is collected by centrifugation at 4000 g for 10 minutes at 4° C., and each pellet is resuspended in 2 mL of 4 M LiCl, mixed well, and re-centrifuged at 4000 g for 10 minutes at 4° C. Each pellet is then resuspended in 2 mL of TE buffer containing 0.5% (w/v) SDS, and an equal volume of chloroform is added. After mixing and centrifugation at 4000 g at 4° C., the upper aqueous phase is removed and the total RNA is precipitated after adding 0.1 volume of 2 M sodium acetate (pH 5.0) and an equal volume of isopropanol. The total RNA is collected by centrifugation at 4000 g for 15 minutes at 4° C. and washed with 70% ethanol and then 100% ethanol. The samples are air dried for 15 minutes and resuspended in 300 mL of sterile DEPC treated water and stored at −80° C. The quality of the RNA is monitored by electrophoresis on a 1.2% (w/v) agarose formaldehyde gel.

Identification of Genes: 10 μg of the total RNA is then used to generate 50-120 μg of amplified antisense RNA, of which 8 μg is used per labeling reaction. Typically, the treated RNA is labeled with Cy-3 and the control RNA is labeled with Cy-5. The two labeled mixtures are then pooled, placed on a whole genome chip and hybridized overnight in custom-made hybridization chambers. Hybridized arrays are washed and scanned with a General Scanning ScanArray 3000, and then quantified using BioDiscovery's Imagene. The quantified field are then normalized and the VOC treated/control expression ratios are determined using in-house developed software and a commercial software from Silicon Genetics, Inc. To ensure meaning results, two repeats for each experiments are carried out. 5-10 top candidate genes will be selected. The criteria for selecting the candidate genes are low background expression before treatment with the VOC, and high-level of induction upon treatment. Genes that are known to be induced by other biotech or abiotic stresses will be excluded.

Validation of Genes

A further goal of these experiments is to verify that the candidate genes are tightly regulated by the VOC. Arabidopsis plants are exposed for one hour, six hours, twelve hours, twenty-four hours and forty-eight hours to benzene and formaldehyde under the same conditions as described above. Leaf tissues are harvested after exposure to VOC. Total RNA from these tissues is isolated and used to analyze the level of mRNA in each treated sample. RT-PCR analysis using I-cycler is used to validate the gene candidates for their induction by the VOC. Oligonucleotides corresponding to the candidate genes are synthesized and used to conduct RT-PCT using I-cycler. The induction kinetics of each of the genes under different treatments and time points is analyzed. These results will allow to further pinpoint the best candidate genes.

Following the selection of promising candidate genes, the 5′ (promoter) of each identified genes are identified; anthocyanin B gene constructs are generated and introduced into Arabidopsis and other indicator plants using Agrobacterium-mediated transformation, and anthocyanin expression is assessed after challenging the plants with VOCs.

Completion of this study will facilitate development of commercial environmental biosensors to detect benzene, formaldehyde and other VOCs, for example, in the air of schools, homes, offices and other environments.

### Example 2

### Identification of Genes Up-Regulated by Heavy Metals

This example describes the use of plant biomarkers to detect heavy metals. For such plant biosensors to be developed, it is important first to identify active metal responsive promoter elements in a plant suitable for use in monitoring systems. In a first step, all of the approximately 25,000 genes of a well-characterized laboratory plant, Arabidopsis thaliana, are screened to identify those genes that are up-regulated by heavy metals.

Identification of Arabidopsis Genes up-regulated by Heavy Metals

Arabidopsis thaliana plants are exposed to different heavy metals, such as arsenic, cadmium, chromium, copper, nickel, mercury, lead, selenium, and zinc. Leaf tissues are then harvested after exposure to heavy metals. Total RNA from these tissues is isolated and used to analyze the level of mRNA in each treated sample. An Affymetric whole genome Arabidopsis micro-array chip, which contains about 25,000 Arabidopsis genes, is used to monitor the expression level of each gene. Hybridization is carried out using a dual color labeling protocol as described above. It is anticipated that hundreds of genes will be identified as up-regulated by each heavy metal. A major milestone will be the identification of a small number (about 5 to 10) of genes that are expressed only in the presence of a heavy metal at the tested concentration.

Arabidopsis plants for heavy metal exposure: Arabidopsis plants are grown as described above.

Exposure of Arabidopsis Plants to Selected Heavy Metals: Initial testing of the response of transformed Arabidopsis plants to heavy metals is done at the seedling stage. Seedlings (3-4 days old) that successfully grew on the selection media (agar containing kanamycin) are transferred to agar plates containing one or more of the metals, arsenic, cadmium, chromium, copper, mercury, nickel, lead, selenium and zinc, at concentrations ranging from 0 to 500 μmol/L. The plants are grown for up to 7 days and monitored for the expression of anthocyanins above background levels. In addition, plants may also be tested at a more mature growth stage. Seedlings (10 days old) are transferred from agar to either a solid media (sand or soil) or solutions, containing one or more of the metals arsenic, cadmium, chromium, copper, mercury, nickel, lead, selenium and zinc at concentrations ranging from 0 to 500 μmol/L (for solution substrates) or 0 to 1000 mg/kg (for solid media). The plants are grown for up to three weeks and evaluated for any potential expression of anthocyanins above that observed in the control treatments. The growth room is maintained at a 16 hours light/8 hours dark, 23° C. regime with light intensity at plant height of 300 μmol m⁻²s⁻¹.

Seven to nine plants are used for treatment and seven to nine plants are used for controls. To ensure the quality and reliability of the experiment, this process is repeated. The plants are then removed, and leaf tissues are placed in liquid nitrogen in preparation for shipment before being analyzed.

Isolation and Analysis of RNA: The leaves from heavy metal treated plants are used for RNA isolation using the hot phenol/lithium chloride method (see, for example, “Current Protocols in Molecular Biology”) and analyzed by gel electrophoresis for quality; and total RNA is isolated and analyzed as described above.

Identification of Genes: Preparation of RNA samples and hybridization are carried out as described above. Hybridized arrays are washed and scanned with a General Scanning ScanArray 3000, and then quantified using BioDiscovery's Imagene. The quantified field are then normalized and the heavy metal treated/control expression ratios are determined using an in-house developed software and a commercial software from Silicon Genetics, Inc. To ensure meaning results, two repeats for each experiments are carried out. 5-10 top candidate genes will be selected. The criteria for selecting the candidate genes are low background expression before treatment with the heavy metal, and high-level of induction upon treatment. Genes that are known to be induced by other biotech or abiotic stresses as well as by non heavy metals will be excluded.

Validation of Genes

A further goal of these experiments is to verify that the candidate genes are tightly regulated by a specific heavy metal. Arabidopsis plants are exposed for one hour, six hours, twelve hours, twenty-four hours and forty-eight hours to arsenic, cadmium, chromium, copper, mercury, nickel, lead, selenium or zinc under the same conditions as described above. Leaf tissues are harvested after exposure to heavy metals. Total RNA from these tissues is isolated and used to analyze the level of mRNA in each treated sample. RT-PCR analysis using 1-cycler is used to validate the gene candidates for their induction by the heavy metal. Oligonucleotides corresponding to the candidate genes are synthesized and used to conduct RT-PCT using I-cycler. The induction kinetics of each of the genes under different treatments and time points is analyzed. These results will allow to further pinpoint the best candidate genes.

Following the selection of promising candidate genes, the 5′ (promoter) of each identified genes are identified; anthocyanin B gene constructs are generated and introduced into Arabidopsis and other indicator plants using Agrobacterium-mediated transformation, and anthocyanin expression is assessed after challenging the plants with heavy metals.

Completion of this study will facilitate development of commercial environmental biosensors for the detection of specific heavy metals.

### Example 3

### Generation of Transgenic Plants for Heavy Metal Detection

This Example and the following one make use of BjMTP (Brassica juncea Metal Tolerance Protein), which has recently been cloned from Indian mustard (B. juncea) seedlings. This gene is thought to be involved in the vacuolar compartmentalization of various metal ions (M. Persans and D. E. Salt, Biotech. Gen. Eng. Rev. 2001, 17: 385-409). Northern analysis revealed that the steady-state expression levels of BjMTP mRNA increases on exposure of Indian mustard seedlings to water containing cadmium (1 μM), nickel (25 μM), or zinc (25 μM). To account for this regulation, it is proposed that the promoter associated with BjMTP contains metal responsive elements (MRE) involved in metal-regulated expression of BjMTP. If the metal regulated expression of BjMTP could be used to regulate expression of a visible feature in plant tissue, such as the B transcription factor that induces anthocyanin synthesis, this metal responsive element will provide a useful tool for the development of “indicator” plants capable of expressing anthocyanins in response to metals.

Generation of Constructs

Constructs are generated in which both the BjMTP1 promoter and the ATMTP1 promoter are fused to the B coding region. Preliminary results have indicated that the ATMTP1 promoter is not inducible by heavy metals. However, it is important to examine the tissue-specificity and developmental expression of this promoter as compared to the basal expression of BJMTP1. The B gene encodes a protein that has been shown to be functionally equivalent to R (S. A. Goff et al., EMBO J. 1990, 9: 2517-2522), which activates the anthocyanin pathway in Arabidopsis when expressed under the control of the 35SCaMV promoter (A. M. Lloyd et al., Science, 1992, 258: 1773-1775). In the experiments presented herein, the B gene is used instead of the R gene because of the availability, in the Applicants laboratory, of a number of B clones and of antibodies that recognize the B protein more efficiently that the R protein (V. L. Chandler, unpublished data).

Additional constructs with each of these promoters are also generated to test their ability to drive B expression in Maize. Maize is a monocot and given the large evolutionary distance between monocots and dicots, these promoters cannot be expected to necessarily function in Maize. However, it is worth noting that there are dicot promoters that function in Maize and vice versa (see, for example, K. Singh et al., Plant Cell, 1990, 2: 891-903; J. Vilardell et al., Plant Mol. Biol. 1994, 24: 561-569; M. L Storgaard et al., Trans. Res. 2002, 11: 151-159; S. Bhattacharyya et al., Virus Res. 2002, 90: 47-62).

For the Arabidopsis experiments, the pCambia BjMTP1promGFP construct, which has previously been prepared by the Applicants, is used.

The steps to construct the Bj::BI plasmid were as follows. The SalI/KpnI fragment of H19 plasmid (which contains Maize AdhI intron I and the 5′ end of the BI genomic sequence, C. Carey and Chandler) was recloned into plasmid pMCG6952 in p_bluescript KS+ bacteria. The Bj promoter sequence was amplified by PCR (for protocol details see Sambrook & Russell, “Molecular Cloning”, 2001, Cold Spring Harbor Laboratory Press). Then pMCG6952 was cut with the restriction enzymes EcoRI and SalI and the amplified Bj promoter sequence was cloned in, to produce the plasmid pMCG7082. In parallel with the above cloning steps, plasmid H19 was digested with restriction enzymes BamHI and EcoRI and the 3′ end of the BI genomic sequence was cloned into the Agrobacterium vector pTF1011, resulting in plasmid pMCG6969. The final step of the procedure was to remove the Bj::AdhI::5′B fragment from plasmid pMCG7082, using restriction enzyme BamHI, and clone this fragment into plasmid pMCG6969, resulting in pMCG7216.

The GFP gene in the pCambia BjMTP1promGFP construct is then replaced with the BcDNA. Examination of the relevant sequences suggests a simple three-step strategy. First, GFP is removed by cutting with two enzymes whose sites flank the GFP coding region and represent unique sites within the vector. Second, the same sites are added to the BcDNA using a convenient shuttle vector. Third, the pCambia BjMTP2prom and BcDNA fragments are ligated.

In the case of the constructs for the generation of transgenic Maize plants, a different vector, pTF101.1 is used. This binary vector has been used for efficient Agrobacterium-mediated transformation of Maize (B. R. Frame et al., Plant Physiol. 2002, 129: 13-22). For unknown reasons, the pCambia vectors do not perform well in Maize transformations, even when engineered to contain identical selectable markers (K. Wang, Iowa Plant Transformation Facility; personal communication). The BjMTP1prom fragment is removed from the pCambia BjMTP1promGTP by digestion with EcoRI and NcoI, and combined with the BcDNA in a shuttle vector. The final step involves removing the now fused BjMTPpromB fragment by digestion with EcoRI and subcloning that fragment directly into pTF101.1. Restriction digestions are conducted at each intermediate step to ensure that the correct cloning occurred, and the final product is sequenced across every site that was manipulated.

Alternatively, the pCambia BjMTP1promGFP construct may be used for the generation of transgenic Maize plants.

Generation of Transgenic Arabidopsis and Maize Plants Containing Intact Constructs

As described above, the regulation of the anthocyanin pathway is well characterized in both Arabidopsis and Maize. Previous experiments have demonstrated that the introduction of the bHLH transcription factor (B or R) can induce anthocyanin biosynthesis in both species in a number of tissues and that the nature of the tissues affected and the amount of pigment generated depend on the promoter used. In the present experiment, the BjMTP1 promoter is tested for its ability to drive anthocyanin expression in these plant species and to induce increased transcription in the presence of heavy metals.

Arabidopsis Transformation: The floral dip method is used for Arabidopsis transformation (S. J. Clough and A. F. Bent, Plant J. 1998, 16: 735-743) and the resulting seeds are plated on kanamycin containing plates to select for primary transformants. Viable seedlings are transferred to soil, their phenotype with respect to pigment is monitored, and resulting plants are allowed to self-fertilize. Transformation is conducted with a positive control construct, in which the 35S CaMV promoter is driving the BcDNA. This construct has been used for Arabidopsis transformation and has resulted in pigmented seedlings (C. Carey and V. L. Chandler, unpublished data).

Maize Transformation: In the production of transgenic Maize plants, the bar gene driven by the Maize ubiquitin promoter is used as a selectable marker (W. J. Gordon-Kamm et al., Plant Cell, 1990, 2: 603-618; A. H. Christensen and P. H. Quail, Transgenic Res. 1996, 5: 213-218). Clones of the construct described above are introduced into Hill, the genotype most often used for Maize transformation, using both bombardment (W. J. Gordon-Kamm et al., Plant Cell, 1990, 2: 603-618) and Agrobacterium-mediated transformation (B. R. Frame et al., Plant Physiol. 2002, 129: 13-22). Callus are initiated from treated embryos in the presence of herbicide and maintained in culture under selection for 8 to 10 weeks. Samples of callus lines that survive selection are then screened for the presence of intact transgene. Positive callus lines are used to regenerate primary transgenic plants (T₀ plants).

The endogenous anthocyanin genes in HiII have been characterized. This background carries functional biosynthetic genes and functional, but weak, alleles of the regulatory genes (p1-sr, a weak pl1 allele that is light inducible, a weak B allele expressed in tassel glumes and the r-r allele expressed in tassel, glume and anthers). Thus, non-transgenic siblings and transformed plants not expressing the introduced B construct should have green leaves and sheaths, pink anthers and pale tassel glumes. If the transgenic T₀ plants produced in the present experiments express the introduced B gene in seedlings in the absence of metal induction (as seen with MTP1 in Bj), anthocyanin accumulation should be observed in seedlings, as the myb factor encoded by p1-sr is expressed there.

The anthocyanin phenotype is monitored in To plants and To plants are crossed with an anthocyanin tester shock that has functional alleles of all the anthocyanin biosynthesis genes, null r and b alleles, and a strong fully functional pl1 allele. The resulting T₁ plants are analyzed for both basal and heavy metal inducible anthocyanin expression. For control purposes, a transgenic line carrying the 35SCaMV promoter fused to BcDNA is also generated. These plants, which express B but are not heavy metal inducible, are darkly pigmented.

Basal Levels of Anthocyanin and Increase in Anthocyanin Accumulation in Transgenic Plants Exposed to Heavy Metals

As described above, anthocyanin accumulation is monitored in all transgenic Arabidopsis and Maize lines grown under standard growth procedures. The threshold and specificity of heavy metals inducing anthocyanin production are evaluated in soil and solution culture. Anthocyanin is monitored visually, the phenotypes recorded by photography, and the amounts of anthocyanin quantified using a spectrophotometer after extraction from ground tissues with an acidic methanol solution (as described in: G. I. Patterson and V. L. Chandler, Maydica, 1995, 40: 35-41). The amounts of B RNA expressed are quantified using Northern blot and RT-PCR procedures and the amounts of B protein are quantified using Western bots (such procedures are, for example, described in: J. P. Radicella et al., Genes & Dev. 1992, 7: 2152-3264).

Threshold Analysis: Seedlings of transformed Arabidopsis plants are analyzed for anthocyanin accumulation in the presence of cadmium, nickel, lead and zinc. Seeds of transformed plants are germinated in 100 mL-beakers containing aerated solutions of 0, 1, 10, 50 or 100 μM of cadmium, nickel, lead, or zinc buffered at pH 6.5. All treatments are conducted using a minimum of three replicates. Upon germination, the seedlings are examined for anthocyanin accumulation daily at intervals for up to 5 days. A subsample is digested and analyzed for metal content to determine the relationship between metal uptake and anthocyanin accumulation. The results of these analyses indicate the response threshold and amplitude of the B transformed seedlings to solution concentrations in a concentration range representative of groundwater, surface waters and soil solution.

Specificity Analysis: The specificity of anthocyanin activity in transformed seedlings is determined using combinations of cadmium, nickel, lead and zinc in conjunction with calcium, iron and magnesium to determine potential interferences of common cations present in water and soil solution. The additive effects of heavy metals on anthocyanin activity are determined in the presence and absence of calcium, magnesium and iron. Seeds of transformed plants are germinated in 100 mL-beakers containing aerated solutions of 0 to 10 mM of cadmium, nickel, lead and zinc, in the presence or absence of 1 mM of calcium or magnesium, or 10 μM of iron. After germination, the seedlings are evaluated for anthocyanin accumulation. The seedlings are transferred to standard nutrient solutions (Hoaglands solution in the presence or absence of cadmium, nickel, lead, or zinc) and evaluated for an additional 14 days.

Anthocyanin Activity in Soil Grown Plants: The accumulation of anthocyanin in soil grown plants is determined by growing transformed seedlings in a soil spiked with cadmium, lead, nickel or zinc. A Sassafras silt loam soil is amended with cadmium (50 mg/kg), lead (400 mg/kg), nickel (400 mg/kg), or zinc (1200 mg/kg) alone and in combination. The soil is limed to pH 6 and equilibrated for three weeks. The soil is then mixed thoroughly. A subsample from each treatment is collected and analyzed for water soluble metal concentrations. The soil is then placed in 8.75 cm diameter pots and seeded with transformed Maize and Arabidopsis. Anthocyanin accumulation is evaluated at 1, 2, 3, 5, 7, 10 and 14 days after emergence to determine the response with plant age. A parallel study is conducted in non-contaminated soil. After 14 days of growth, the non-contaminated soil is spiked with cadmium, lead, nickel and zinc and the plants are grown for an additional seven days to assess response to applied metals. Plant samples are then analyzed for metal concentrations in the shoots.

Comparison of BIMTP1GFP and BIMTP1B Arabidopsis Plants: Arabidopsis plants transformed with BjMTP1B and Arabidopsis plants transformed with BJM1GFP are exposed to heavy metals and their responses are compared. Seeds are germinated in potting mix and transferred to 8.75 cm diameter pots. After 14 days of growth, the pots are treated with solutions of 0, 10 or 20 μM cadmium, nickel, or zinc. The intensity of fluorescence under UV light of GFP-transformed plants is compared with accumulation of anthocyanins in B transformed plants at 1, 2, 3, 5, and 10 days after exposure to determine potential effectiveness of the two biosensing techniques in response to heavy metal exposure.

Conclusion

These procedures will provide the basis for field application of plant biosensors. Insertion of genetic constructs will first be performed using annual and perennial crop species that can be used to monitor large soil areas. Crops such as turf grass or other perennial grasses have increased utility to monitor contaminated soils in high exposure areas because of their longevity and adaptability to a wide variety of growing conditions, particularly in the residential sector. Sensitivity, duration, visibility and response to anthocyanin accumulation will then be studied as a function of plant growth stage and specific metal contaminant. Assessment of the performance and utility of the biosensing crop will culminate in a field study.

### Example 4

### Transgenic Plants as Biomarkers for Heavy Metals in Drinking Water

The procedures described herein are designed to determine the feasibility of using plant biomarkers to detect arsenic, cadmium, and lead in drinking water. The expression of the BjMRP mRNA is studied when Brassica juncea seedlings are exposed to arsenic, cadmium, and lead at varying concentrations and durations and in combination with other competing metals and ions found in drinking water (e.g., chloride, fluoride, iron, calcium, and magnesium).

Expression of BjMTP mRNA in Brassica juncea Seedlings

Brassica juncea seeds are germinated by placing the seeds in opaque plastic trays with water and aerating the solutions. Three-day old Brassica juncea seedlings are exposed to solutions containing AsO₃²⁻, Cd²⁺, or Pb²⁺ at concentrations in the range near recommended drinking water limits to levels found in contaminated water systems (0, 0.1, 0.5, 1, 10 and 20 μM). This concentration range is selected to help determine if the expression of BjMTP is concentration dependent at the magnitude of drinking water MCL's (parts per billion range). The seedlings are exposed to the aerated solutions for 24, 48 and 72 hours in a darkened cell at 20° C. The seedlings are then removed from the solutions, rinsed with distilled water. The seedlings may then be analyzed by RNA extraction and Northern analysis and for total metal content.

Effects of Oxidation State of Arsenic: Three-day old Brassica juncea seedlings are exposed to solutions containing 20 μM AsO₃²⁻. The seedlings are exposed for 48 hours in a darkened cell at 20° C. The seedlings are then removed from the solutions, rinsed with distilled water and analyzed by RNA extraction and Northern analysis and for total metal content.

Effects of Competing Metals or Ions: Three-day old Brassica juncea seedlings are placed in aerated distilled water and solutions containing 20 μM As(III), 1 μM Cd²⁺, or 20 μM Pb²⁺ in the presence of 10, 50 or 100 μM Fe³⁺, or 0.5, 1.0 or 10 μM Ca²⁺, Mg²⁺, Cl⁻ or F⁻. The seedlings are exposed for 48 hours in a darkened cell at 20° C. The seedlings are then removed, rinsed with distilled water and analyzed by RNA extraction and Northern analysis and for total metal content.

Total Metal Analysis

Plant samples are dried at 70° C., ground to pass a 20 mesh screen using a stainless steel Wiley Mill and digested using nitric acid and hydrogen peroxide (EPA Method 3050). The resulting solution is analyzed for total metals using inductively coupled plasma optical emission spectroscopy.

RNA Extraction and Analysis of BjMTP mRNA Expression

Total RNA is isolated following the method reported by C. Puissant and L. M. Houbebine (Biotech. 1990, 8: 148-149)). Approximately 5 to 10 g of Brassica juncea seedlings are ground to a fine powder in liquid nitrogen in a chilled mortar and pestle. The ground tissue is placed in 4 to 6 Falcon 2059 centrifuge tubes (Becton Dickinson, Lincoln Park, N.J.) each containing 5 mL of GuISCN extraction buffer (4 M guanidinium isothiocyanate, 25 mM sodium citrate (pH 7.0), 05% (w/v) N-lauroyl sarkosine and 0.1 M β-mercaptoethanol), and the tubes are mixed by inversion.

Samples are mixed with 0.1 volume of 1 M sodium acetate (pH 4.0) and 5 mL of phenol:chloroform (5:1) are added. Samples are then mixed and centrifuged at 5000 g for 15 minutes at 4° C. The aqueous phase (about 7 mL) is removed and placed in a fresh 15 mL Falcon 2059 tube and the RNA is precipitated by addition of an equal volume of isopropanol at 4° C. The RNA is collected by centrifugation at 4000 g for 10 minutes at 4° C., and each pellet is suspended in 2 mL of 4 M LiCl, mixed well, and re-centrifuged at 4000 g for 10 minutes at 4° C. Each pellet is then resuspended in 2 mL of TE buffer containing 0.5% (w/v) SDS, and an equal volume of chloroform is added. After mixing and centrifugation at 4000 g at 4° C., the upper aqueous phase is removed and the total RNA is precipitated after adding 0.1 volume of 2 M sodium acetate (pH 5.0) and an equal volume of isopropanol. The total RNA is collected by centrifugation at 4000 g for 15 minutes at 4° C. and washed with 70% ethanol and then 100% ethanol. The samples are air dried for 15 minutes and resuspended in 300 mL of sterile DEPC treated water and stored at −80° C.

For Northern analysis, 30 μg of total RNA is electrophoresed on 1.2% (w/v) agarose formaldehyde gels and capillary-blotted overnight onto nylon membranes (Pharmacia Biotech), using 10×SSC. The RNA is UV-crosslinked to the membrane and the blot prehybridized in 10 mL of a pre-hybridization solution containing 200 mM Na₂PO₄ (pH 7.2), 5% SD, 1 mM EDTA, 10 mg/mL BSA, and 0.1 mg/mL sheared salmon sperm DNA for at least 2 hours at 65° C. The blots are then probed with denatured α-³²P-labeled probe added directly to the hybridization solution and incubated at 65° C. for 12 to 16 hours. The blots are washed twice for 15-20 minutes at 65° C. with 50 mL of a solution containing 40 mM Na₂PO₄ (pH 7.2), 5% SDS, 1 mM EDTA, 5 mg/mL BSA. Blots are then washed for a second time in 50 mL of a solution containing 40 mM of Na₂PO₄ (pH 7.2), 1% SDS, 1 mM EDTA, for 30-35 minutes at 60 to 65° C. After washing, the blots are placed on a phosphorimager screen and read using the phosphorimager after 12 to 24 hours. As an RNA loading control blots are then stripped and re-probed with an actin probe.

For cDNA probe preparation, one hour restriction digests of pTrip1Ex-[BjMTP] with EcoRI and XhoI, and an Arabidopsis thaliana actin gene (Genbank accession # U37281) with BamHI and EcoTI are performed at 37° C. The resulting fragments are run on a 1.5% agarose gel and the appropriate size fragment excised from the gel and recovered by electro-elution. In advance of blot hybridization, 75-150 ng of DNA (about 5 μL) are denatured at 100° C. for 10 minutes in 35 mL of ddH₂O, and the sample is snap cooled on ice for 30 seconds. Two μL BSA (10 mg/mL), 10 μL of 5× OLB buffer (250 mM Tris HCl (pH 8.0), 25 mM MgCl₂, 0.35% β-mercaptoethanol, 100 μM dGTP, dCTP, and dTTP, 1 M HEPES (pH 6.0) and 0.54 μg/μL pdN6 random hexamers), 2-3 mL of α-³²P dATP (10 μCi/μL), and 5 units of DNA polymerase I Klenow fragment added to the denatured DNA. The labeling reaction is incubated for at least five hours at room temperature. The labeled DNA probe is boiled for 10 minutes, snap cooled on ice for 30 seconds, centrifuged for 10 seconds at 14,000 g and added directly to the hybridization buffer.

Conclusion

The next steps will consists of efforts to isolate the putative metal responsive promoter (MRP) element from Indian mustard and generate an MRP-B construct to be stably expressed in Arabidopsis thaliana via Agrobacterium mediated germ-line transformation. Once created, these transgenic Arabidopsis seedlings will be tested for metal regulated expression of anthocyanin. These efforts will be followed by developing Indian mustard that is stably transformed with the MRP-B construct. The plant biosensors will then be characterized for their ability to detect arsenic, cadmium, and lead concentrations in drinking water at both the laboratory and field levels, and pilot-scale field trials will be conducted to test for real-time water monitoring.

1. A nucleic acid construct comprising:
a gene encoding a product involved in the biosynthesis of a plant pigment, said gene being operably linked to an inducible sensor system, wherein the sensor system is responsive to the presence of a contaminant.

2. The construct of claim 1, wherein the gene encodes a product involved in the biosynthesis of anthocyanins.

3. The construct of claim 2, wherein the gene encodes an anthocyanin regulatory gene B, an anthocyanin regulatory gene R, or translational equivalents thereof.

4. The construct of claim 1, wherein the sensor system comprises a contaminant-responsive promoter element.

5. The construct of claim 1, wherein the contaminant comprises an inorganic compound or element.

6. The construct of claim 5, wherein the contaminant comprises a metal or metalloid.

7. The construct of claim 6, wherein the contaminant comprises a heavy metal selected from the group consisting of antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium and zinc.

8. The construct of claim 5, wherein the contaminant comprises an inorganic anion selected from the group consisting of chloride, perchlorate, nitrate, phosphate and sulfate.

9. The construct of claim 5, wherein the contaminant comprises a gaseous inorganic compound (GIC).

10. The construct of claim 9, wherein the GIC is selected from the group consisting of ozone, a sulfur oxide (SOx), and a nitrogen oxide (NOx).

11. The construct of claim 1, wherein the contaminant comprises an organic compound.

12. The construct of claim 11, wherein the contaminant comprises a volatile organic compound (VOC).

13. The construct of claim 12, wherein the VOC is benzene or formaldehyde.

14. The construct of claim 11, wherein the contaminant comprises a halogenated organic compound (HOC).

15. The construct of claim 14, wherein the HOC is selected from the group consisting of a polychlorinated biphenyl (PCB), a polychlorinated hydrocarbon (PCH), a chlorofluorocarbon (Freon), and a chlorofluorohydrocarbon.

16. The construct of claim 11, wherein the contaminant is an organic compound selected from the group consisting of a dioxin, a furan, a polycyclic aromatic hydrocarbon, and an ether.

17. The construct of claim 1, wherein the contaminant comprises a pesticide.

18. The construct of claim 1, wherein the contaminant comprises an agent of chemical or biological warfare.

19. The construct of claim 18, wherein the agent is selected from the group consisting of sarin, ricin, mustard gas, phosgene, anthrax bacilli, anthrax spores, and smallpox virus.

20. The construct of claim 1, wherein induction of the sensor system causes expression of the gene in response to the presence of the contaminant.

21. The construct of claim 1, wherein induction of the sensor system causes increased expression of the gene in response to the presence of the contaminant.

22. The construct of claim 21, wherein the increase in expression of the gene is proportional to the concentration of contaminant present.

23. The construct of claim 21, wherein the increase in expression of the gene is proportional to the concentration of bioavailable contaminant present.

24. The construct of claim 1 further comprising a sequence selected from the group consisting of a leader sequence, an intron sequence, a transcription terminator, a polyadenylation site, and any combinations thereof.

25. The construct of claim 1 further comprising a sequence selected from the group consisting of a marker gene sequence, a selectable gene sequence, a T-DNA sequence, and any combinations thereof.

26. A transgenic plant cell comprising a transgene comprising:
a gene encoding a product involved in the biosynthesis of a plant pigment, said gene being operably linked to an inducible sensor system, wherein the sensor system is responsive to the presence of a contaminant.

27. The transgenic plant cell of claim 26, wherein the cell is from a monocotyledonous plant species.

28. The transgenic plant cell of claim 26, wherein the cell is from a dicotyledonous plant species.

29. The transgenic plant cell of claim 26, wherein the cell is from a plant selected from the group consisting of turfgrasses, members of the Brassicaceae family, ferns of the Pteris genus of the Pteridaceae family, members of the Nicotiana genus of the Solanacea family, and members of the Poaceae family.

30. The transgenic plant cell of claim 26, wherein the cell is from Maize or an Arabidopsis.

31. The transgenic plant cell of claim 26, wherein the cell is from a crop plant, a landscaping plant or a house plant.

32. The transgenic plant cell of claim 26, wherein the gene encodes a product involved in the biosynthesis of anthocyanins.

33. The transgenic plant cell of claim 32, wherein the gene encodes an anthocyanin regulatory gene B, an anthocyanin regulatory gene R, or translational equivalents thereof.

34. The transgenic plant cell of claim 26, wherein the sensor system comprises a contaminant-responsive promoter element.

35. The transgenic plant cell of claim 26, wherein the contaminant comprises an inorganic compound or element.

36. The transgenic plant cell of claim 35, wherein the contaminant comprises a metal or metalloid.

37. The transgenic plant cell of claim 36, wherein the contaminant comprises a heavy metal selected from the group consisting of antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium, and zinc.

38. The transgenic plant cell of claim 35, wherein the contaminant comprises an inorganic anion or a gaseous inorganic compound.

39. The transgenic plant cell of claim 26, wherein the contaminant comprises an organic compound.

40. The transgenic plant cell of claim 39, wherein the contaminant comprises a volatile organic compound (VOC).

41. The transgenic plant cell of claim 40, wherein the VOC is benzene or formaldehyde.

42. The transgenic plant cell of claim 39, wherein the contaminant comprises a halogenated organic compound (HOC).

43. The transgenic plant cell of claim 26, wherein the contaminant comprises a pesticide.

44. The transgenic plant cell of claim 26, wherein the contaminant comprises an agent of chemical or biological warfare.

45. The transgenic plant cell of 26, wherein induction of the sensor system causes expression of the gene in response to the presence of the contaminant.

46. The transgenic plant cell of claim 26, wherein induction of the sensor system causes increased expression of the gene in response to the presence of the contaminant.

47. The transgenic plant cell of claim 46, wherein the increase in expression of the gene is proportional to the concentration of contaminant present.

48. The transgenic plant cell of claim 46, wherein the increase in expression of the gene is proportional to the concentration of bioavailable contaminant present.

49. A transgenic plant exhibiting a change in color phenotype when exposed to a contaminant, the transgenic plant comprising a transgene comprising:
a gene encoding a product involved in the biosynthesis of a plant pigment, said gene being operably linked to an inducible sensor system which is responsive to the presence of a contaminant.

50. The transgenic plant cell of claim 49, wherein the plant is a monocotyledonous plant.

51. The transgenic plant cell of claim 49, wherein the plant is a dicotyledonous plant.

52. The transgenic plant of claim 49, wherein the plant is selected from the group consisting of turfgrasses, members of the Brassicaceae family, ferns of the Pteris genus of the Pteridaceae family, members of the Nicotiana genus of the Solanacea family, and members of the Poaceae family.

53. The transgenic plant of claim 49, wherein the plant is Maize or an Arabidopsis.

54. The transgenic plant of claim 49, wherein the plant is a crop plant, a landscaping plant or a house plant.

55. The transgenic plant of claim 49, wherein the gene encodes a product involved in the biosynthesis of anthocyanins.

56. The transgenic plant of claim 55, wherein the gene encodes an anthocyanin regulatory gene B, an anthocyanin regulatory gene R, or translational equivalents thereof.

57. The transgenic plant of claim 49, wherein the sensor system comprises a contaminant-responsive promoter element.

58. The transgenic plant of claim 49, wherein the contaminant comprises an inorganic compound or element.

59. The transgenic plant of claim 58, wherein the contaminant comprises a metal or metalloid.

60. The transgenic plant of claim 59, wherein the contaminant comprises a heavy metal selected from the group consisting of antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium, and zinc.

61. The transgenic plant of claim 58, wherein the contaminant comprises an inorganic anion or a gaseous inorganic compound.

62. The transgenic plant of claim 49, wherein the contaminant comprises an organic compound.

63. The transgenic plant of claim 62, wherein the contaminant comprises a volatile organic compound (VOC).

64. The transgenic plant of claim 63, wherein the VOC is benzene or formaldehyde.

65. The transgenic plant of claim 62, wherein the contaminant comprises a halogenated organic compound (HOC).

66. The transgenic plant of claim 49, wherein the contaminant comprises a pesticide.

67. The transgenic plant cell of claim 49, wherein the contaminant comprises an agent of chemical or biological warfare.

68. The transgenic plant of claim 49, wherein exposure of the transgenic plant to the contaminant causes induction of the sensor system resulting in expression of the gene and a change in color phenotype of the plant.

69. The transgenic plant of claim 49, wherein exposure of the transgenic plant to the contaminant causes induction of the sensor system resulting in increased expression of the gene and a change in color phenotype of the plant.

70. The transgenic plant of claim 69, wherein the increase in expression of the gene is proportional to the concentration of contaminant present.

71. The transgenic plant of claim 69, wherein the increase in expression of the gene is proportional to the concentration of bioavailable contaminant present.

72. A seed of any generation of the transgenic plant of claim 49, wherein the seed comprises said transgene.

73. A progeny of the transgenic plant of claim 49, wherein the plant progeny comprises said transgene.

74. Plant material from the transgenic plant of claim 49, wherein the plant material comprises said transgene.

75. A transgenic plant exhibiting different changes in color phenotype when exposed to different contaminants, the transgenic plant comprising more than one transgene, each transgene comprising:
a gene encoding a product involved in the biosynthesis of a plant pigment, said gene being operably linked to an inducible sensor system which is responsive to the presence of a particular contaminant and whose induction by that particular contaminant is associated with a specific change in color phenotype.

76. A method for preparing a transgenic plant exhibiting a change in color phenotype when exposed to a contaminant, the method comprising:
introducing a nucleic acid construct into a plant cell or protoplast to obtain a stably transformed plant cell or protoplast, the construct comprising:
a gene encoding a product involved in the biosynthesis of a plant pigment, said gene being operably linked to an inducible sensor system, wherein the sensor system is responsive to the presence of the contaminant;
regenerating a whole plant from the stably transformed plant cell or protoplast to obtain a transgenic plant comprising a transgene comprising:
a gene encoding a product involved in the biosynthesis of a plant pigment, said gene being operably linked to an inducible sensor system, wherein the sensor system is responsive to the presence of the contaminant; and
exposing the transgenic plant to the contaminant.

77. The method of claim 76, wherein the contaminant is present in the air, in water or in a soil.

78. The method of claim 76, wherein the plant cell or protoplast is from a monocotyledonous plant species.

79. The method of claim 76, wherein the plant cell or protoplast is from a dicotyledonous plant species.

80. The method of claim 76, wherein the plant cell or protoplast is from a plant selected from the group consisting of turfgrasses, members of the Brassicaceae family, ferns of the Pteris genus of the Pteridaceae family, members of the Nicotiana genus of the Solanacea family, and members of the Poaceae family.

81. The method of claim 76, wherein the plant cell is from a crop plant, a landscaping plant or a house plant.

82. A stably transformed plant cell obtained by the method of claim 76.

83. A stably transformed protoplast obtained by the method of claim 76.

84. A transgenic plant obtained by the method of claim 76.

85. A seed of any generation of a transgenic plant obtained by the method of claim 76, wherein the seed comprises said transgene.

86. A progeny of a transgenic plant obtained by the method of claim 76, wherein the plant progeny comprises said transgene.

87. Plant material from a transgenic plant obtained by the method of claim 76, wherein the plant material comprises said transgene.

88. The method of claim 76, wherein the gene encodes a product involved in the biosynthesis of anthocyanins.

89. The method of claim 88, wherein the gene encodes an anthocyanin regulatory gene B, an anthocyanin regulatory gene R, or translational equivalents thereof.

90. The method of claim 76, wherein the sensor system comprises a contaminant-responsive promoter element.

91. The method of claim 76, wherein the contaminant comprises an inorganic compound or element.

92. The method of claim 91, wherein the contaminant comprises a metal or metalloid.

93. The method of claim 92, wherein the contaminant comprises a heavy metal selected from the group consisting of antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium, and zinc.

94. The method of claim 91, wherein the contaminant comprises an inorganic anion or a gaseous inorganic compound.

95. The method of claim 76, wherein the contaminant comprises an organic compound.

96. The method of claim 95, wherein the contaminant comprises a volatile organic compound (VOC).

97. The method of claim 96, wherein the VOC is benzene or formaldehyde.

98. The method of claim 95, wherein the contaminant comprises a halogenated organic compound (HOC).

99. The method of claim 76, wherein the contaminant comprises a pesticide.

100. The method of claim 76, wherein the contaminant comprises an agent of chemical or biological warfare.

101. The method of claim 76, wherein exposure of the plant to the contaminant induces the sensor system causing expression of the gene and a change in color phenotype of the plant.

102. The method of claim 76, wherein exposure of the plant to the contaminant induces the sensor system causing increased expression of the gene and a change in color phenotype of the plant.

103. The method of claim 102, wherein the increase in expression of the gene is proportional to the concentration of contaminant present.

104. The method of claim 102, wherein the increase in expression of the gene is proportional to the concentration of bioavailable contaminant present.

105. A method for detecting the presence of a contaminant in an area, the method comprising:
introducing, in the area to be tested, seeds from a transgenic plant comprising a transgene comprising:
a gene encoding a product involved in the biosynthesis of a plant pigment, said gene being operably linked to an inducible sensor system, wherein the sensor system is responsive to the presence of the contaminant;
growing transgenic plants from the seeds;
monitoring the color phenotype of the resulting plants; and
determining that the contaminant is present in the area tested if a change in color phenotype was observed in the resulting plants.

106. The method of claim 105, wherein introducing seeds comprises planting the seeds in the soil of the area to be tested.

107. The method of claim 105, wherein introducing seeds comprises planting the seeds in potting soil or growth media to obtain seedlings, and introducing the seedlings to the area to be tested.

108. The method of claim 105, wherein the transgenic plants are grown in water.

109. The method of claim 105, wherein the contaminant is present in the air, in water or in a soil.

110. The method of claim 105, wherein the seeds are from a monocotyledonous plant species.

111. The method of claim 105, wherein the seeds are from a dicotyledonous plant species.

112. The method of claim 105, wherein the seeds are from a plant selected from the group consisting of turfgrasses, members of the Brassicaceae family, ferns of the Pteris genus of the Pteridaceae family, members of the Nicotiana genus of the Solanacea family, and members of the Poaceae family.

113. The method of claim 105, wherein the seeds are from a crop plant, a landscaping plant or a house plant.

114. The method of claim 105, wherein the gene encodes a product involved in the biosynthesis of anthocyanins.

115. The method of claim 114, wherein the gene encodes an anthocyanin regulatory gene B, an anthocyanin regulatory gene R, or translational equivalents thereof.

116. The method of claim 105, wherein the sensor system comprises a contaminant-responsive promoter element.

117. The method of claim 105, wherein the contaminant comprises an inorganic compound or element.

118. The method of claim 117, wherein the contaminant comprises a metal or metalloid.

119. The method of claim 118, wherein the contaminant comprises a heavy metal selected from the group consisting of antimony, arsenic, bismuth, cadmium, cerium, cesium, chromium, cobalt, copper, gallium, gold, iron, lead, manganese, mercury, nickel, platinum, silver, strontium, tellurium, thallium, tin, uranium, vanadium, and zinc.

120. The method of claim 117, wherein the contaminant comprises an inorganic anion or a gaseous inorganic compound.

121. The method of claim 105, wherein the contaminant comprises an organic compound.

122. The method of claim 121, wherein the contaminant comprises a volatile organic compound (VOC).

123. The method of claim 122, wherein the VOC is benzene or formaldehyde.

124. The method of claim 121, wherein the contaminant comprises a halogenated organic compound (HOC).

125. The method of claim 105, wherein the contaminant comprises a pesticide.

126. The method of claim 105, wherein the contaminant comprises an agent of chemical or biological warfare.

127. The method of claim 105, wherein presence of the contaminant in the area tested induces the sensor system causing expression of the gene and a change in color phenotype of the resulting plants.

128. The method of claim 105, wherein presence of the contaminant in the area tested induces the sensor system causing increased expression of the gene and a change in color phenotype of the resulting plants.

129. The method of claim 128, wherein the increase in expression of the gene is proportional to the concentration of contaminant present in the area tested, and wherein the method further comprises assessing the intensity of change in color phenotype of the resulting plants; and, based on the intensity, determining the concentration of contaminant present in the area.

130. The method of claim 128, wherein the increase in expression of the gene is proportional to the concentration of bioavailable contaminant present in the area tested, and wherein the method further comprises assessing the intensity of change in color phenotype of the resulting plants; and, based on the intensity, determining the concentration of bioavailable contaminant present in the area.

131. A method for detecting the presence of contaminants in an area, the method comprising:
introducing, in the area to be tested, seeds from a transgenic plant comprising more than one transgene, each transgene comprising:
a gene encoding a product involved in the biosynthesis of a plant pigment, said gene being operably linked to an inducible sensor system which is responsive to the presence of a particular contaminant and whose induction by that particular contaminant is associated with a specific change in color phenotype;
growing transgenic plants from the seeds;
monitoring the color phenotype of the resulting plants; and
determining that a particular contaminant is present in the area tested if the associated change in color phenotype was observed in the resulting plants.
